SEQUENCE LISTING (53 sequences)

SEQ ID NO: 1 - protein, 115 aa, synthetic construct
QVQLVQSGAEVKKPGSSVKVSCKASGGAFSSYAISWVRQAPGQGLEWMGRIIPILGIANYAQKFQGRVTITADKSTSTAYMELSSLRSEDTAVYYCAGGSGYYYWGQGTTVTVSS

SEQ ID NO: 2 - protein, 216 aa, synthetic construct
QSVLTQPPSVSEAPGQRVTISCSGSSSNIANNAVNWYQQLPGQAPKLLIFYDDLLPSWVSDRFSGSKSGTSASLAISGLQSEDEADYYCAAWDDSLNGPVFGGGTKLTVLRQPVAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPAECS

SEQ ID NO: 3 - protein, 7 aa, synthetic construct
GGAFSSY

SEQ ID NO: 4 - protein, 6 aa, synthetic construct
IPILGI

SEQ ID NO: 5 - protein, 6 aa, synthetic construct
GSGYYY

SEQ ID NO: 6 - protein, 13 aa, synthetic construct
SGSSSNIANNAVN

SEQ ID NO: 7 - protein, 7 aa, synthetic construct
YDDLLPS

SEQ ID NO: 8 - protein, 11 aa, synthetic construct
AAWDDSLNGPV

SEQ ID NO: 9 - protein, 113 aa, synthetic construct
QVQLQQWGAGLLKPSETLSLTCAVYGGSFSGNYWSWIRQPPGKGLEWIGEINHSGSTNYNPSLKSRVTISVDTSKNQFSLKLSSVTAADTAVYYCARGLLDYWGQGTLVTVSS

SEQ ID NO: 10 - protein, 216 aa, synthetic construct
QSVLTQPSSVSAAPWQRVTISCSGSDSNIGNNPVNWYQHVPGKAPTLLIYYDNFLASKISDRFSGSKSGTSASLAISGLQSDDEADYYCASWDDSLNGVVFGGGTKLTVLRQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 11 - protein, 7 aa, synthetic construct
GGSFSGN

SEQ ID NO: 12 - protein, 5 aa, synthetic construct
NHSGS

SEQ ID NO: 13 - protein, 5 aa, synthetic construct
GLLDY

SEQ ID NO: 14 - protein, 13 aa, synthetic construct
SGSDSNIGNNPVN

SEQ ID NO: 15 - protein, 7 aa, synthetic construct
YDNFLAS

SEQ ID NO: 16 - protein, 11 aa, synthetic construct
ASWDDSLNGVV

SEQ ID NO: 17 - protein, 114 aa, synthetic construct
QVQLVQSGAEVKKPGASVKVSCKASGYTFTGYYMHWVRQAPGQGLEWMGRINPNSGGTNYAQKFQGRVAMTRDTSISTAYMELSRLRSDDTAVYYCARGYMDYWGQGTPVTVSS

SEQ ID NO: 18 - protein, 216 aa, synthetic construct
QSVLTQPPSASGTPGQRVTISCSGSSSNIGSSTVNWYQQLPETAPKLLIYSNHQRPSGVPDRFSGSKSGTSASLALSGLQSEDEAEYYCAAWDDSLNGVVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 19 - protein, 7 aa, synthetic construct
GYTFTGY

SEQ ID NO: 20 - protein, 6 aa, synthetic construct
NPNSGG

SEQ ID NO: 21 - protein, 5 aa, synthetic construct
GYMDY

SEQ ID NO: 22 - protein, 13 aa, synthetic construct
SGSSSNIGSSTVN

SEQ ID NO: 23 - protein, 7 aa, synthetic construct
SNHQRPS

SEQ ID NO: 24 - protein, 11 aa, synthetic construct
AAWDDSLNGVV

SEQ ID NO: 25 - protein, 330 aa, synthetic construct [sequence omitted]

SEQ ID NO: 26 - protein, 20 aa, synthetic construct
METDTLLLWVLLLWVPGSTG

SEQ ID NO: 27 - protein, 18 aa, synthetic construct
GSTSGSGKPGSGEGSTKG

SEQ ID NO: 28 - protein, 20 aa, synthetic construct
GGGGSGGGGSGGGGSGGGGS

SEQ ID NO: 29 - protein, 707 aa, synthetic construct
QVQLQQWGAGLLKPSETLSLTCAVYGGSFSGNYWSWIRQPPGKGLEWIGEINHSGSTNYNPSLKSRVTISVDTSKNQFSLKLSSVTAADTAVYYCARGLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGSTSGSGKPGSGEGSTKGQSVLTQPPSVSEAPGQRVTISCSGSSSNIANNAVNWYQQLPGQAPKLLIFYDDLLPSWVSDRFSGSKSGTSASLAISGLQSEDEADYYCAAWDDSLNGPVFGGGTKLTVLGGGGGSGGGGSGGGGSGGGGSQVQLVQSGAEVKKPGSSVKVSCKASGGAFSSYAISWVRQAPGQGLEWMGRIIPILGIANYAQKFQGRVTITADKSTSTAYMELSSLRSEDTAVYYCAGGSGYYYWGQGTTVTVSSA

SEQ ID NO: 30 - protein, 247 aa, synthetic construct
QSVLTQPPSVSEAPGQRVTISCSGSSSNIANNAVNWYQQLPGQAPKLLIFYDDLLPSWVSDRFSGSKSGTSASLAISGLQSEDEADYYCAAWDDSLNGPVFGGGTKLTVLGGGGGSGGGGSGGGGSGGGGSQVQLVQSGAEVKKPGSSVKVSCKASGGAFSSYAISWVRQAPGQGLEWMGRIIPILGIANYAQKFQGRVTITADKSTSTAYMELSSLRSEDTAVYYCAGGSGYYYWGQGTTVTVSSA

SEQ ID NO: 31 - protein, 708 aa, synthetic construct
QVQLVQSGAEVKKPGSSVKVSCKASGGAFSSYAISWVRQAPGQGLEWMGRIIPILGIANYAQKFQGRVTITADKSTSTAYMELSSLRSEDTAVYYCAGGSGYYYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSQSVLTQPSSVSAAPWQRVTISCSGSDSNIGNNPVNWYQHVPGKAPTLLIYYDNFLASKISDRFSGSKSGTSASLAISGLQSDDEADYYCASWDDSLNGVVFGGGTKLTVLGGGGSGGGGSGGGGSGGGGSQVQLQQWGAGLLKPSETLSLTCAVYGGSFSGNYWSWIRQPPGKGLEWIGEINHSGSTNYNPSLKSRVTISVDTSKNQFSLKLSSVTAADTAVYYCARGLLDYWGQGTLVTVSSA

SEQ ID NO: 32 - protein, 244 aa, synthetic construct
QSVLTQPSSVSAAPWQRVTISCSGSDSNIGNNPVNWYQHVPGKAPTLLIYYDNFLASKISDRFSGSKSGTSASLAISGLQSDDEADYYCASWDDSLNGVVFGGGTKLTVLGGGGSGGGGSGGGGSGGGGSQVQLQQWGAGLLKPSETLSLTCAVYGGSFSGNYWSWIRQPPGKGLEWIGEINHSGSTNYNPSLKSRVTISVDTSKNQFSLKLSSVTAADTAVYYCARGLLDYWGQGTLVTVSSA

SEQ ID NO: 33 - protein, 120 aa, synthetic construct
QVQLVQSGAEVKKPGSSVKVSCKASGGTFSSYAISWVRQAPGQGLEWMGGIIPILGIANYAQKFQGRVTITADKSTSTAYMELSSLRSEDTAVYYCARDGGGSSGYHDYWGQGTLVTVSS

SEQ ID NO: 34 - protein, 216 aa, synthetic construct
NFMLTQPHSVSESPGKTVTISCTRSSGSIASKYVQWYQQRPGNSPTTVIYEDDQRPSGVPDRFSASIDSSANTASLTISGLKPEDEADYYCQSYDDNTAVFGGGTHLTVLGQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 35 - protein, 7 aa, synthetic construct
GGTFSSY

SEQ ID NO: 36 - protein, 6 aa, synthetic construct
IPILGI

SEQ ID NO: 37 - protein, 11 aa, synthetic construct
DGGGSSGYHDY

SEQ ID NO: 38 - protein, 13 aa, synthetic construct
TRSSGSIASKYVQ

SEQ ID NO: 39 - protein, 7 aa, synthetic construct
EDDQRPS

SEQ ID NO: 40 - protein, 9 aa, synthetic construct
QSYDDNTAV

SEQ ID NO: 41 - protein, 127 aa, synthetic construct
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCATQTPYYDSSDYYLGDAFDIWGQGTMVTVSS

SEQ ID NO: 42 - protein, 216 aa, synthetic construct
QSALTQPASVSGSPGQSITISCTGTSSDVGGYNYVSWYQQHPGKAPKLIIYDVRNRPSGVSNRFSGSKSGNTASLTISGLQAEDEADYYCGSYTSSSTLGFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 43 - protein, 7 aa, synthetic construct
GYSFTSY

SEQ ID NO: 44 - protein, 6 aa, synthetic construct
YPGDSD

SEQ ID NO: 45 - protein, 18 aa, synthetic construct
QTPYYDSSDYYLGDAFDI

SEQ ID NO: 46 - protein, 14 aa, synthetic construct
TGTSSDVGGYNYVS

SEQ ID NO: 47 - protein, 7 aa, synthetic construct
DVRNRPS

SEQ ID NO: 48 - protein, 10 aa, synthetic construct
GSYTSSSTLG

SEQ ID NO: 49 - protein, 713 aa, synthetic construct
QVQLVQSGAEVKKPGSSVKVSCKASGGTFSSYAISWVRQAPGQGLEWMGGIIPILGIANYAQKFQGRVTITADKSTSTAYMELSSLRSEDTAVYYCARDGGGSSGYHDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSQSVLTQPSSVSAAPWQRVTISCSGSDSNIGNNPVNWYQHVPGKAPTLLIYYDNFLASKISDRFSGSKSGTSASLAISGLQSDDEADYYCASWDDSLNGVVFGGGTKLTVLGGGGSGGGGSGGGGSGGGGSQVQLQQWGAGLLKPSETLSLTCAVYGGSFSGNYWSWIRQPPGKGLEWIGEINHSGSTNYNPSLKSRVTISVDTSKNQFSLKLSSVTAADTAVYYCARGLLDYWGQGTLVTVSSA

SEQ ID NO: 50 - protein, 714 aa, synthetic construct
QVQLVQSGAEVKKPGSSVKVSCKASGGTFSSYAISWVRQAPGQGLEWMGGIIPILGIANYAQKFQGRVTITADKSTSTAYMELSSLRSEDTAVYYCARDGGGSSGYHDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSQSVLTQPPSASGTPGQRVTISCSGSSSNIGSSTVNWYQQLPETAPKLLIYSNHQRPSGVPDRFSGSKSGTSASLALSGLQSEDEAEYYCAAWDDSLNGVVFGGGTKLTVLGGGGSGGGGSGGGGSGGGGSQVQLVQSGAEVKKPGASVKVSCKASGYTFTGYYMHWVRQAPGQGLEWMGRINPNSGGTNYAQKFQGRVAMTRDTSISTAYMELSRLRSDDTAVYYCARGYMDYWGQGTPVTVSSA

SEQ ID NO: 51 - protein, 720 aa, synthetic construct
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCATQTPYYDSSDYYLGDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSQSVLTQPSSVSAAPWQRVTISCSGSDSNIGNNPVNWYQHVPGKAPTLLIYYDNFLASKISDRFSGSKSGTSASLAISGLQSDDEADYYCASWDDSLNGVVFGGGTKLTVLGGGGSGGGGSGGGGSGGGGSQVQLQQWGAGLLKPSETLSLTCAVYGGSFSGNYWSWIRQPPGKGLEWIGEINHSGSTNYNPSLKSRVTISVDTSKNQFSLKLSSVTAADTAVYYCARGLLDYWGQGTLVTVSSA

SEQ ID NO: 52 - protein, 721 aa, synthetic construct
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCATQTPYYDSSDYYLGDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSQSVLTQPPSASGTPGQRVTISCSGSSSNIGSSTVNWYQQLPETAPKLLIYSNHQRPSGVPDRFSGSKSGTSASLALSGLQSEDEAEYYCAAWDDSLNGVVFGGGTKLTVLGGGGSGGGGSGGGGSGGGGSQVQLVQSGAEVKKPGASVKVSCKASGYTFTGYYMHWVRQAPGQGLEWMGRINPNSGGTNYAQKFQGRVAMTRDTSISTAYMELSRLRSDDTAVYYCARGYMDYWGQGTPVTVSSA

SEQ ID NO: 53 - protein, 245 aa, synthetic construct
QSVLTQPPSASGTPGQRVTISCSGSSSNIGSSTVNWYQQLPETAPKLLIYSNHQRPSGVPDRFSGSKSGTSASLALSGLQSEDEAEYYCAAWDDSLNGVVFGGGTKLTVLGGGGSGGGGSGGGGSGGGGSQVQLVQSGAEVKKPGASVKVSCKASGYTFTGYYMHWVRQAPGQGLEWMGRINPNSGGTNYAQKFQGRVAMTRDTSISTAYMELSRLRSDDTAVYYCARGYMDYWGQGTPVTVSSA

(19) United States
(12) Patent Application Publication
(10) Pub. No.: US 2026/0250363 A1
(43) Pub. Date: Aug. 27, 2026

(54) SARS-CoV-2 ANTIBODIES AND BISPECIFIC ANTIBODIES

(71) Applicant: AP Biosciences, Inc., Taipei City (TW)

(72) Inventors: Jeng-Horng Her, San Jose, CA (US); Ching-Hsuan Hsu, Taoyuan City (TW); Ting-Yi Chang, New Taipei City (TW); Jhong-Jhe You, Taipei City (TW)

(21) Appl. No.: 18/992,869

(22) PCT Filed: Jul. 26, 2023
(86) PCT No.: PCT/US2023/028677

§ 371 Date: Jan. 9, 2025

Related U.S. Application Data
(60) Provisional application No. 63/392,466, filed on Jul. 26, 2022.

(51) Int. Cl. C07K 16/104; A61P 31/14

(52) U.S. Cl. CPC C07K 16/104; A61P 31/14; C07K 2317/31; C07K 2317/622

(57) ABSTRACT

Provided herein are antibodies or antigen binding fragments thereof having a binding specificity for SARS-CoV-2 spike protein, and bispecific antibodies comprising a first and a second antigen binding region that bind to SARS-CoV-2 spike protein. Also provided herein are pharmaceutical compositions comprising the antibodies, antigen binding fragments thereof, or bispecific antibodies.

### CROSS-REFERENCE TO RELATED APPLICATIONS

This application claims benefit of priority under 35 U.S.C. § 119(e) of U.S. Provisional Application No. 63/392,466, filed Jul. 26, 2022. The disclosure of the prior application is considered part of and is herein incorporated by reference in the disclosure of this application in its entirety.

### INCORPORATION OF SEQUENCE LISTING

The material in the accompanying sequence listing is hereby incorporated by reference into this application. The accompanying sequence listing xml file, name 7-21-23-AP1120-1WO_SL, was created on Jul. 21, 2023 and is 53 kb.

### BACKGROUND OF THE INVENTION

### Field of the Invention

The present invention relates generally to antibodies and antigen binding fragments and more specifically to antibodies and antigen binding fragments for neutralizing SARS-CoV-2 variants.

### Background Information

In December 2019, pneumonia of unknown cause was identified in clusters of patients in Wuhan City, China. Later, a newly emergent coronavirus was identified in these patients. Due to its potent transmission rate, the coronavirus that causes severe acute respiratory syndrome swift spread in China and around the world. In early 2020, the World Health Organization (WHO) announced that this was a Public Health Emergency of International Concern (PHEIC), and formally called it Coronavirus Disease-2019 (COVID-19). The International Committee on Taxonomy of Viruses named the virology as severe acute respiratory syndrome coronavirus 2 (SARS-CoV-2). By April 2022, more than 490 million confirmed cases of COVID-19 have been reported globally.

While vaccines remain the best strategy to prevent COVID-19, antibodies could potentially benefit certain vulnerable populations, such as the unvaccinated or vaccinated high-risk patients. Monoclonal antibodies (mAbs) that can bind to and neutralize the virus are a novel class of antiviral intervention (Simeonov, A. et., al., 2020; Phoolcharoen, W. et., al., 2020). Cocktails of two or more mAbs are preferred over a single antibody as they result in increased efficacy and the prevention of rapid mutational escape. However, this approach still poses challenges in mitigating future rapid mutational escape and increases manufacturing cost and volumes, which are problematic at a time when the supply chain is under pressure to meet COVID-19 therapeutics, vaccines, and other biologics in general (Ecker, D. M. & Seymour, P., 2020).

In this patent, several novel monoclonal antibodies against the spike proteins of SARS-CoV-2 variants were generated and screened upon the binding and neutralizing activity. Simultaneously, various antibody pairs were selected and transformed into a bispecific antibody format to broaden and intensify the neutralizing activity against SARS-CoV-2 variants. Ultimately, bispecific antibodies showed a greatly enhancement in neutralizing SARS-CoV-2 Omicron pseudo virus compared to the combination of its parental monoclonal antibodies.

### SUMMARY OF THE INVENTION

The present invention is based on the discovery of anti-SARS-CoV-2 spike protein antibodies that possess neutralizing activity against different strains of SARS-CoV-2. The present invention is further based on the discovery of bispecific antibodies derived from two distinct anti-SARS-CoV-2 spike protein antibody clones that possess neutralizing activity against the known SARS-CoV-2 variants, including original strain, Alpha strain, Lambda strain, Delta strain, Delta plus strain, Omicron strain, and Omicron BA.2 sub strain.

In some embodiments, the present invention provides five antibodies or antigen binding fragment thereof: anti-SARS-CoV-2 antibody clone R4-11 (R4-11), anti-SARS-CoV-2 antibody clone R4-1a-10 (R4-1a-10), anti-SARS-CoV-2 antibody clone R4-1a-51 (R4-1a-51), anti-SARS-CoV-2 antibody clone R3-1a-1 (R3-1a-1) and anti-SARS-CoV-2 antibody clone R4-21 (R4-21). In one aspect, anti-SARS-CoV-2 antibodies provided herein include a heavy chain variable (VH) region including an amino acid sequence with at least 80% identity to a sequence selected from SEQ ID NO:1, SEQ ID NO:9, SEQ ID NO:17, SEQ ID NO:33 and SEQ ID NO:41; and a light chain variable (VL) region including an amino acid sequence with at least 80% identity to a sequence selected from SEQ ID NO:2, SEQ ID NO:10, SEQ ID NO:18, SEQ ID NO:34 and SEQ ID NO:42.

In one aspect, the antibody or antigen binding fragment having a VH region that includes an amino acid sequence having at least 80% identity to SEQ ID NO:1 and a VL region that includes an amino acid sequence having at least 80% identity to SEQ ID NO:2 includes (a) VH CDR-H1, CDR-H2, and CDR-H3, wherein CDR-H1 includes an amino acid sequence having at least 80% identity to SEQ ID NO:3, wherein CDR-H2 includes an amino acid sequence having at least 80% identity to SEQ ID NO:4, and wherein CDR-H3 includes an amino acid sequence having at least 80% identity to SEQ ID NO:5; and (b) VL CDR-L1, CDR-L2, and CDR-L3, wherein CDR-L1 includes an amino acid sequence having at least 80% identity to SEQ ID NO:6, wherein CDR-L2 includes an amino acid sequence having at least 80% identity to SEQ ID NO:7, and wherein CDR-L3 includes an amino acid sequence having at least 80% identity to SEQ ID NO:8.

In another aspect, the antibody or antigen binding fragment thereof having a VH region that includes an amino acid sequence having at least 80% identity to SEQ ID NO:9 and a VL region that includes an amino acid sequence having at least 80% identity to SEQ ID NO:10 includes (a) VH CDR-H1, CDR-H2, and CDR-H3, wherein CDR-H1 includes an amino acid sequence having at least 80% identity to SEQ ID NO:11, wherein CDR-H2 includes an amino acid sequence having at least 80% identity to SEQ ID NO:12, and wherein CDR-H3 includes an amino acid sequence having at least 80% identity to SEQ ID NO:13; and (b) VL CDR-L1, CDR-L2, and CDR-L3, wherein CDR-L1 includes an amino acid sequence having at least 80% identity to SEQ ID NO:14, wherein CDR-L2 includes an amino acid sequence having at least 80% identity to SEQ ID NO:15, and wherein CDR-L3 includes an amino acid sequence having at least 80% identity to SEQ ID NO:16.

In one aspect, the antibody or antigen binding fragment thereof having a VH region that includes an amino acid sequence having at least 80% identity to SEQ ID NO:17 and a VL region that includes an amino acid sequence having at least 80% identity to SEQ ID NO:18 includes (a) VH CDR-H1, CDR-H2, and CDR-H3, wherein CDR-H1 includes an amino acid sequence having at least 80% identity to SEQ ID NO:19, wherein CDR-H2 includes an amino acid sequence having at least 80% identity to SEQ ID NO:20, and wherein CDR-H3 includes an amino acid sequence having at least 80% identity to SEQ ID NO:21; and (b) VL CDR-L1, CDR-L2, and CDR-L3, wherein CDR-L1 includes an amino acid sequence having at least 80% identity to SEQ ID NO:22, wherein CDR-L2 includes an amino acid sequence having at least 80% identity to SEQ ID NO:23, and wherein CDR-L3 includes an amino acid sequence having at least 80% identity to SEQ ID NO:24.

In another aspect, the antibody or antigen binding fragment thereof having a VH region that includes an amino acid sequence having at least 80% identity to SEQ ID NO:33 and a VL region that includes an amino acid sequence having at least 80% identity to SEQ ID NO:34 includes (a) VH CDR-H1, CDR-H2, and CDR-H3, wherein CDR-H1 includes an amino acid sequence having at least 80% identity to SEQ ID NO:35, wherein CDR-H2 includes an amino acid sequence having at least 80% identity to SEQ ID NO:36, and wherein CDR-H3 includes an amino acid sequence having at least 80% identity to SEQ ID NO:37; and (b) VL CDR-L1, CDR-L2, and CDR-L3, wherein CDR-L1 includes an amino acid sequence having at least 80% identity to SEQ ID NO:38, wherein CDR-L2 includes an amino acid sequence having at least 80% identity to SEQ ID NO:39, and wherein CDR-L3 includes an amino acid sequence having at least 80% identity to SEQ ID NO:40.

In one aspect, the antibody or antigen binding fragment thereof having a VH region that includes an amino acid sequence having at least 80% identity to SEQ ID NO:41 and a VL region that includes an amino acid sequence having at least 80% identity to SEQ ID NO:42 includes (a) VH CDR-H1, CDR-H2, and CDR-H3, wherein CDR-H1 includes an amino acid sequence having at least 80% identity to SEQ ID NO:43, wherein CDR-H2 includes an amino acid sequence having at least 80% identity to SEQ ID NO:44, and wherein CDR-H3 includes an amino acid sequence having at least 80% identity to SEQ ID NO:45; and (b) VL CDR-L1, CDR-L2, and CDR-L3, wherein CDR-L1 includes an amino acid sequence having at least 80% identity to SEQ ID NO:46, wherein CDR-L2 includes an amino acid sequence having at least 80% identity to SEQ ID NO:47, and wherein CDR-L3 includes an amino acid sequence having at least 80% identity to SEQ ID NO:48.

In one aspect, antibodies or antigen binding fragments thereof provided herein include an Fc domain. In another aspect, the Fc domain is an IgG, IgE, IgM, IgD, IgA, or IgY domain. In yet another aspect, the IgG domain is an IgG1, IgG2, IgG3, or IgG4 domain. In an additional aspect, the IgG1 domain includes an amino acid sequence of SEQ ID NO:25.

In another embodiment, the invention provides a bispecific antibody including a first antigen binding region that binds to a first SARS-CoV-2 spike protein antigen and a second antigen binding region that binds to a second SARS-CoV-2 spike protein antigen. In one aspect, the bispecific antibody has the amino acid sequence as set forth in SEQ ID NO:29, 31, 49, 50, 51 or 52, or a sequence having 90% identity thereto and the binding specificity thereof. In one aspect, bispecific antibodies provided herein include an Fc domain. In another aspect, the Fc domain is an IgG domain, an IgE domain, an IgM domain, and IgD domain, an IgA domain, or an IgY domain. In yet another aspect, the Fc domain is an IgG domain. In a further aspect, the IgG domain is an IgG1 domain, an IgG2 domain, an IgG3 domain, or an IgG4 domain.

In one aspect, the scFv is linked to the C-terminus of the Fc domain. In another aspect, bispecific antibodies provided herein include a linker between the Fab domain and the scFv domain. In a further aspect, the Fab fragment is linked to the N-terminus of the Fc domain.

In an embodiment, the present disclosure also provides a pharmaceutical composition that includes any one of the antibodies or an antigen binding fragment thereof provided herein or any one of the bispecific antibodies provided herein. In one aspect, antibodies or antigen binding fragments or bispecific antibodies of the pharmaceutical compositions provided herein include a pharmaceutically acceptable carrier conjugated to the C-terminus of one or more polypeptides of the antibody or antigen binding fragment thereof.

In one embodiment, the invention provides an isolated amino acid sequence as set forth in SEQ ID NOs:1-53.

In one embodiment, the present disclosure also provides an isolated nucleic acid sequence encoding the antibody, an antigen-binding fragment thereof, or the bispecific antibody of the invention. In another embodiment, the invention provides an isolated nucleic acid encoding any one of SEQ ID NOs:1-53.

### BRIEF DESCRIPTION OF THE DRAWINGS

FIGS. 1A-1H show the screening for phage clones targeted to SARS-CoV-2 by ELISA. The phage clones recognize SARS-CoV-2 RBD specifically after four rounds of enrichment process from OmniMab phage library. Phage-containing supernatant were applied to SARS-CoV-2 ELISA, and positive binders were numbered. FIG. 1A is a graph showing ELISA results of phage clones isolated from fourth-round panning, screening by SARS-CoV-2 spike RBD variants, included WT, E484K N501Y, and E484K alone. FIG. 1B is a graph showing ELISA results of phage clones isolated from fourth-round panning, screening by SARS-CoV-2 spike RBD variants, included WT, E484K N501Y, and E484K alone. FIG. 1C is a graph showing ELISA results of phage clones isolated from fourth-round panning, screening by SARS-CoV-2 spike RBD variants, included L452R E484Q, and L452R T478K. FIG. 1D is a graph showing ELISA results of phage clones isolated from fourth-round panning, screening by SARS-CoV-2 spike RBD variants, included L452R E484Q, and L452R T478K. FIG. 1E is a graph showing ELISA results of phage clones isolated from fourth-round panning, screening by SARS-CoV-2 spike RBD variants, included L452R E484Q, and L452R T478K. FIG. 1F is a graph showing ELISA results of phage clones isolated from fourth-round panning, screening by SARS-CoV-2 spike RBD variants, included WT, E484K N501Y, E484K alone, L452R E484Q, and L452R T478K. FIG. 1G is a graph showing ELISA results of phage clones isolated from third-round panning. FIG. 1H is a table showing a list of clone name numbered in FIG. 1A to FIG. 1G.

FIGS. 2A-2E show the purity of protein A-purified anti-SARS-CoV-2 antibody leads by SDS-PAGE. The purity and integrity of one-step Protein A-purified anti-SARS-CoV-2 antibody leads was analyzed by SDS-PAGE under non-reducing and reducing conditions. FIG. 2A shows a photograph of an SDS-PAGE for clones 1-9 in non-reduced (left) and reduced (right) conditions. FIG. 2B shows a photograph of an SDS-PAGE for clones 10-17 in non-reduced (left) and reduced (right) conditions. FIG. 2C shows a photograph of an SDS-PAGE for clones 18-24 in non-reduced (left) and reduced (right) conditions. FIG. 2D shows a photograph of an SDS-PAGE for clones 25-27 in non-reduced (left) and reduced (right) conditions. FIG. 2E shows a photograph of an SDS-PAGE for the lead antibodies.

FIGS. 3A-3G show binding activity of anti-SARS-CoV-2 antibody leads by direct ELISA. FIG. 3A is a graph showing single dose binding activity of SARS-CoV-2 spike protein Omicron variants. FIG. 3B is a graph showing anti-SARS-CoV-2 antibody leads binding with SARS-CoV-2 spike protein wild type. FIG. 3C is a graph showing anti-SARS-CoV-2 antibody leads binding with SARS-CoV-2 spike protein alpha strain. FIG. 3D is a graph showing anti-SARS-CoV-2 antibody leads binding with SARS-CoV-2 spike protein lambda strain. FIG. 3E is a graph showing anti-SARS-CoV-2 antibody leads binding with SARS-CoV-2 spike protein delta strain. FIG. 3F is a graph showing anti-SARS-CoV-2 antibody leads binding with SARS-CoV-2 spike protein delta plus strain. FIG. 3G is a graph showing anti-SARS-CoV-2 antibody leads binding with SARS-CoV-2 spike protein omicron strain.

FIGS. 4A-4G show neutralizing activity of anti-SARS-CoV-2 antibody leads by competition ELISA. FIG. 4A is a graph showing inhibition of SARS-CoV-2 spike protein variants-hACE2 interaction by anti-SARS-CoV-2 antibodies single dose. FIG. 4B is a graph showing inhibition of SARS-CoV-2 spike protein-hACE2 interaction by anti-SARS-CoV-2 antibodies. FIG. 4C is a graph showing inhibition of SARS-CoV-2 spike protein alpha strain-hACE2 interaction by anti-SARS-CoV-2 antibodies. FIG. 4D is a graph showing inhibition of SARS-CoV-2 spike protein lambda strain-hACE2 interaction by anti-SARS-CoV-2 antibodies. FIG. 4E is a graph showing inhibition of SARS-CoV-2 spike protein delta strain-hACE2 interaction by anti-SARS-CoV-2 antibodies. FIG. 4F is a graph showing inhibition of SARS-CoV-2 spike protein delta plus strain-hACE2 interaction by anti-SARS-CoV-2 antibodies. FIG. 4G is a graph showing inhibition of SARS-CoV-2 spike protein omicron strain-hACE2 interaction by anti-SARS-CoV-2 antibodies.

FIG. 5 shows the symmetric format of an anti-SARS-CoV-2 bispecific antibody (bsAb).

FIGS. 6A-6K show the neutralizing activity of anti-SARS-CoV-2 bispecific antibody leads by competition ELISA. FIG. 6A is a graph showing inhibition of SARS-CoV-2 spike protein hACE2 interaction by anti-SARS-CoV-2 bispecific antibodies. FIG. 6B is a graph showing inhibition of SARS-CoV-2 spike protein variant alpha hACE2 interaction by anti-SARS-CoV-2 bispecific antibodies. FIG. 6C is a graph showing inhibition of SARS-CoV-2 spike protein variant lambda hACE2 interaction by anti-SARS-CoV-2 bispecific antibodies. FIG. 6D is a graph showing inhibition of SARS-CoV-2 spike protein variant delta hACE2 interaction by anti-SARS-CoV-2 bispecific antibodies. FIG. 6E is a graph showing inhibition of SARS-CoV-2 spike protein variant delta plus hACE2 interaction by anti-SARS-CoV-2 bispecific antibodies. FIG. 6F is a graph showing inhibition of SARS-CoV-2 spike protein variant omicron hACE2 interaction by anti-SARS-CoV-2 bispecific antibodies. FIG. 6G is a graph showing binding activity of anti-SARS-CoV-2 bispecific antibody to SARS-CoV-2 spike protein with Omicron variants by direct ELISA with a single dose binding activity of SARS-CoV-2 spike protein Omicron variants. FIG. 6H is a graph showing anti-SARS-CoV-2 neutralizing bispecific antibodies blocking the SARS-CoV-2 spike protein from binding to hACE2 receptor proteins through inhibition of SARS-CoV-2 spike protein variants-hACE2 interaction by anti-SARS-CoV-2 bispecific antibodies. FIG. 6I is a graph showing anti-SARS-CoV-2 neutralizing bispecific antibodies blocking the SARS-CoV-2 spike protein from binding to hACE2 receptor proteins through inhibition of SARS-CoV-2 spike protein variants-hACE2 interaction by anti-SARS-CoV-2 bispecific antibodies. FIG. 6J is a graph showing anti-SARS-CoV-2 neutralizing bispecific antibodies blocking the SARS-CoV-2 spike protein from binding to hACE2 receptor proteins through inhibition of SARS-CoV-2 spike protein variants-hACE2 interaction by anti-SARS-CoV-2 bispecific antibodies. FIG. 6K is a graph showing anti-SARS-CoV-2 neutralizing bispecific antibodies blocking the SARS-CoV-2 spike protein from binding to hACE2 receptor proteins through inhibition of SARS-CoV-2 spike protein variants-hACE2 interaction by anti-SARS-CoV-2 bispecific antibodies.

FIGS. 7A-7B show neutralization of the SARS-CoV-2 Omicron strain pseudo virus by anti-SARS-CoV-2 combination monoclonal antibodies and bispecific antibody. Infection of 293T-hACE2 cells by SARS-CoV-2 Omicron pseudo virus was determined in combination of monoclonal antibodies and bispecific antibodies. Luciferase activities in the 293T-hACE2 cells were measured, and the percent inhibition (%) was calculated. Data are presented as means in duplicates. FIG. 7A is a graph showing the results of the neutralization assay for two antibodies in comparison with imdevimab+ casirivimab. FIG. 7B is a graph showing the results of the neutralization assay for four antibodies in comparison with cilgavimab+ tixagevimab.

FIGS. 8A-8J show the protein aggregation of anti-SARS-CoV-2 bsAb by SEC-HPLC with different protein concentrate. FIG. 8A is a graph illustrating analysis of anti-SARS-CoV-2 Ra-1a-1/R4-11 scFv bsAb protein aggregation by SEC-HPLC. FIG. 8B is a graph illustrating analysis of anti-SARS-CoV-2 Ra-1a-1/R4-11 scFv bsAb protein aggregation by SEC-HPLC. FIG. 8C is a graph illustrating analysis of anti-SARS-CoV-2 Ra-1a-1/R4-11 scFv bsAb protein aggregation by SEC-HPLC. FIG. 8D is a graph illustrating analysis of anti-SARS-CoV-2 Ra-1a-1/R4-11 scFv bsAb protein aggregation by SEC-HPLC. FIG. 8E is a table summarizing data in FIGS. 8A-8D. FIG. 8F is a graph illustrating analysis of anti-SARS-CoV-2 R3-1a-1/R4-1a-10 scFv bsAb protein aggregation by SEC-HPLC. FIG. 8G is a graph illustrating analysis of anti-SARS-CoV-2 R3-1a-1/R4-1a-10 scFv bsAb protein aggregation by SEC-HPLC. FIG. 8H is a graph illustrating analysis of anti-SARS-CoV-2 R3-1a-1/R4-1a-10 scFv bsAb protein aggregation by SEC-HPLC. FIG. 8I is a graph illustrating analysis of anti-SARS-CoV-2 R3-1a-1/R4-1a-10 scFv bsAb protein aggregation by SEC-HPLC. FIG. 8J is a table summarizing the data in FIG. 8F-8I.

FIGS. 9A-9D show the protein integrity of anti-SARS-CoV-2 bsAb by μCE-SDS-PAGE. FIG. 9A is a graph illustrating SDS-PAGE data for R4-1a-10/R4-11 scFv in non-reducing conditions. FIG. 9B SDS-PAGE data for R4-1a-10/R4-11 scFv in reducing conditions. FIG. 9C is a graph illustrating SDS-PAGE data for R3-1a-1/R4-1a-10 scFv in non-reducing conditions. FIG. 9D SDS-PAGE data for R3-1a-1/R4-1a-10 scFv in reducing conditions.

### DETAILED DESCRIPTION OF THE INVENTION

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

Provided herein, in some embodiments, are antibodies and antigen binding fragments thereof that bind SARS-CoV-2 spike protein. Also provided herein are amino sequences of antibodies that bind SARS-CoV-2 spike protein. As used herein, the term “antibody” refers to an immunoglobulin molecule that has the ability to specifically bind to an antigen. The term “antibody” included, but is not limited to, monoclonal antibodies, polyclonal antibodies, human antibodies, humanized antibodies, chimeric antibodies, bispecific antibodies, and anti-isotypic antibodies, unless context clearly indicates otherwise. In one aspect, antibodies provided herein include monoclonal antibodies. Antibodies provided herein include any isotype and class (e.g., IgG, IgE, IgM, IgD, IgA and IgY), or subclass (e.g., IgG1, IgG2, IgG3, IgG4, IgA1 and IgA2). As used herein, “antigen binding fragment” means a fragment or portion of an immunoglobulin molecule or antibody that has the ability to specifically bind to the same antigen as the immunoglobulin molecule or antibody. Exemplary antigen binding fragments include scFv, Fab, or F(ab)2 fragments. As used herein, “antigen binding region” means the part of an antibody or immunoglobulin molecule that binds to antigens or proteins by contacting the antigen or protein, for example. An antigen binding region generally includes heavy chain variable (VH) regions and light chain variable (VL) regions. An antigen binding region generally includes one or more antigen binding sites or paratopes.

Antibodies provided herein have a VH region including an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to a sequence of SEQ ID NO:1, SEQ ID NO:9, SEQ ID NO:17, SEQ ID NO:33, or SEQ ID NO:41. Antibodies provided herein also include an VL region that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to a sequence of SEQ ID NO:2, SEQ ID NO:10, SEQ ID NO:18, SEQ ID NO:34, or SEQ ID NO:42.

In general, “sequence identity” or “sequence homology,” which can be used interchangeably, refer to an exact nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby or the amino acid sequence of a polypeptide and comparing these sequences to a second nucleotide or amino acid sequence. As used herein, the term “percent (%) sequence identity” or “percent (%) identity,” also including “homology,” refers to the percentage of amino acid residues or nucleotides in a sequence that are identical with the amino acid residues or nucleotides in a reference sequence after aligning the sequences and introducing gaps, if necessary, to achieve the maximum percent sequence identity, and not considering any conservative substitutions as part of the sequence identity. Thus, two or more sequences (polynucleotide or amino acid) can be compared by determining their “percent identity,” also referred to as “percent homology.” The percent identity to a reference sequence (e.g., nucleic acid or amino acid sequences), which may be a sequence within a longer molecule (e.g., polynucleotide or polypeptide), may be calculated as the number of exact matches between two optimally aligned sequences divided by the length of the reference sequence and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA 87:2264-2268 (1990) and as discussed in Altschul, et al., J. Mol. Biol. 215:403-410 (1990); Karlin and Altschul, Proc. Natl. Acad. Sci. USA 90:5873-5877 (1993); and Altschul et al., Nucleic Acids Res. 25:3389-3402 (1997). Briefly, the BLAST program defines identity as the number of identical aligned symbols (i.e., nucleotides or amino acids), divided by the total number of symbols in the shorter of the two sequences. The program may be used to determine percent identity over the entire length of the sequences being compared. Default parameters are provided to optimize searches with short query sequences, for example, with the blastp program. The program also allows use of an SEG filter to mask-off segments of the query sequences as determined by the SEG program of Wootton and Federhen, Computers and Chemistry 17: 149-163 (1993). Ranges of desired degrees of sequence identity are approximately 80% to 100% and integer values in between. Percent identities between a reference sequence and a claimed sequence can be at least 80%, at least 85%, at least 90%, at least 95%, at least 98%, at least 99%, at least 99.5%, or at least 99.9%. In general, an exact match indicates 100% identity over the length of the reference sequence. Additional programs and methods for comparing sequences and/or assessing sequence identity include the Needleman-Wunsch algorithm (see, e.g., the EMBOSS Needle aligner available at www.ebi.ac.uk/Tools/psa/emboss_needle/, optionally with default settings), the Smith-Waterman algorithm (see, e.g., the EMBOSS Water aligner available at www.ebi.ac.uk/Tools/psa/emboss_water/, optionally with default settings), the similarity search method of Pearson and Lipman, 1988, Proc. Natl. Acad. Sci. USA 85, 2444, or computer programs which use these algorithms (GAP, BESTFIT, FASTA, BLAST P, BLAST N and TFASTA in Wisconsin Genetics Software Package, Genetics Computer Group, 575 Science Drive, Madison, Wis.). In some aspects, reference to percent sequence identity refers to sequence identity as measured using BLAST (Basic Local Alignment Search Tool). In other aspects, ClustalW is used for multiple sequence alignment. Optimal alignment may be assessed using any suitable parameters of a chosen algorithm, including default parameters.

The terms “sequence identity” or “percent identity” are used interchangeably herein. To determine the percent identity of two polypeptide molecules or two polynucleotide sequences, the sequences are aligned for optimal comparison purposes (e.g., gaps can be introduced in the sequence of a first polypeptide or polynucleotide for optimal alignment with a second polypeptide or polynucleotide sequence). The amino acids or nucleotides at corresponding amino acid or nucleotide positions are then compared. When a position in the first sequence is occupied by the same amino acid or nucleotide as the corresponding position in the second sequence, then the molecules are identical at that position. The percent identity between the two sequences is a function of the number of identical positions shared by the sequences (i.e., % identity=number of identical positions/total number of positions (i.e., overlapping positions)×100). In some embodiments the length of a reference sequence aligned for comparison purposes is at least 80% of the length of the comparison sequence, and in some embodiments is at least 90% or 100%. In an embodiment, the two sequences are the same length.

Ranges of desired degrees of sequence identity are approximately 80% to 100% and integer values in between. Percent identities between a disclosed sequence and a claimed sequence can be at least 80%, at least 85%, at least 90%, at least 95%, at least 96%, at least 97%, at least 98%, at least 99%, at least 99.5%, or at least 99.9%. In general, an exact match indicates 100% identity over the length of the reference sequence. Polypeptides and polynucleotides that are about 85, 86, 87, 88, 89, 90, 91, 92, 93, 94, 95, 96, 97, 98, 99 99.5% or more identical to polypeptides and polynucleotides described herein are embodied within the disclosure.

Variants of the disclosed sequences also include peptides, or full-length protein, which contain substitutions, deletions, or insertions into the protein backbone, which would still leave at least about 70% homology to the original protein over the corresponding portion. A yet greater degree of departure from homology is allowed if like-amino acids, i.e., conservative amino acid substitutions, do not count as a change in the sequence. Examples of conservative substitutions involve amino acids that have the same or comparable properties. Illustrative amino acid conservative substitutions include the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine; threonine to serine; tryptophan to tyrosine; tyrosine to tryptophan or phenylalanine; valine to isoleucine to leucine.

In one aspect, the antibody or antigen binding fragment thereof has a VH region that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:1 and a VL region that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:2. SEQ ID NO:1 provides an amino acid sequence that includes the variable region of anti-SARS-CoV-2 antibody clone R4-11 heavy chain. SEQ ID NO: 2 provides an amino acid sequence that includes the variable region of anti-SARS-CoV-2 antibody clone R4-11 light chain.

The antigen binding region of an antibody or antigen binding fragment thereof generally includes complementarity determining regions (CDRs). “Complementarity determining region (CDR)” refers to hypervariable regions of VH and VL. CDRs include the target protein or antigen binding site of an antibody that confers specificity for protein or antigen binding. VH and VL generally include three sequentially numbered CDRs. As used herein, CDR-H1, CDR-H2, and CDR-H3 refer to three consecutively arranged CDRs of the heavy chain variable region (VH), as numbered from the N-terminus of the heavy chain polypeptide. As used herein, CDR-L1, CDR-L2, and CDR-L3 refer to three consecutively arranged CDRs of the light chain variable region (VL), as numbered from the N-terminus of the light chain polypeptide.

In one aspect, the antigen binding region of the antibody or antigen binding fragment thereof having a VH region that includes an amino acid sequence having at least about 80% identity to SEQ ID NO:1 and a VL region that includes an amino acid sequence having at least about 80% identity to SEQ ID NO:2 has a CDR-H1 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:3, a CDR-H2 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:4, and a CDR-H3 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:5. In another aspect, the antigen binding region of the antibody or antigen binding fragment thereof having a VH region that includes an amino acid sequence having at least about 80% identity to SEQ ID NO:1 and a VL region that includes an amino acid sequence having at least about 80% identity to SEQ ID NO:2 has a CDR-L1 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:6, a CDR-L2 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:7, and a CDR-L3 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:8.

In some aspects, the antibody or antigen binding fragment thereof includes a VH region that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:9 and a VL region that includes an amino acid sequence having at least about 80%, identity at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:10. SEQ ID NO:9 provides an amino acid sequence that includes the variable region of anti-SARS-CoV-2 antibody clone R4-1a-10 heavy chain. SEQ ID NO:10 provides an amino acid sequence that includes the variable region of anti-SARS-CoV-2 antibody clone R4-1a-10 light chain.

In one aspect, the antigen binding region of the antibody or antigen binding fragment thereof that includes a VH region including an amino acid sequence having at least about 80% identity to SEQ ID NO:9 and a VL region including an amino acid sequence having at least about 80% identity to SEQ ID NO:10 has a CDR-H1 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:11, a CDR-H2 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:12, and a CDR-H3 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:13. In another aspect, the antigen binding region of the antibody or antigen binding fragment thereof that includes a VH region including an amino acid sequence having at least about 80% identity to SEQ ID NO:9 and a VL region that includes an amino acid sequence having at least about 80%, identity to SEQ ID NO:10 has a CDR-L1 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:14, a CDR-L2 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:15, and a CDR-L3 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:16.

In some aspects, the antibody or antigen binding fragment thereof includes a VH region that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:17 and a VL region that includes an amino acid sequence having at least about 80%, identity at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:18. SEQ ID NO:17 provides an amino acid sequence that includes the variable region of anti-SARS-CoV-2 antibody clone R4-1a-51 heavy chain. SEQ ID NO: 18 provides an amino acid sequence that includes the variable region of anti-SARS-CoV-2 antibody clone R4-1a-51 light chain.

In one aspect, the antigen binding region of the antibody or antigen binding fragment thereof that includes a VH region including an amino acid sequence having at least about 80% identity to SEQ ID NO:17 and a VL region including an amino acid sequence having at least about 80% identity to SEQ ID NO:18 has a CDR-H1 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:19, a CDR-H2 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:20, and a CDR-H3 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:21. In another aspect, the antigen binding region of the antibody or antigen binding fragment thereof that includes a VH region including an amino acid sequence having at least about 80% identity to SEQ ID NO:17 and a VL region that includes an amino acid sequence having at least about 80%, identity to SEQ ID NO:18 has a CDR-L1 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:22, a CDR-L2 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:23, and a CDR-L3 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:24.

In some aspects, the antibody or antigen binding fragment thereof includes a VH region that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:33 and a VL region that includes an amino acid sequence having at least about 80%, identity at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:34. SEQ ID NO:33 provides an amino acid sequence that includes the variable region of anti-SARS-CoV-2 antibody clone R3-1a-1 heavy chain. SEQ ID NO: 34 provides an amino acid sequence that includes the variable region of anti-SARS-CoV-2 antibody clone R3-1a-1 light chain.

In one aspect, the antigen binding region of the antibody or antigen binding fragment thereof that includes a VH region including an amino acid sequence having at least about 80% identity to SEQ ID NO:33 and a VL region including an amino acid sequence having at least about 80% identity to SEQ ID NO:34 has a CDR-H1 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:35, a CDR-H2 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:36, and a CDR-H3 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:37. In another aspect, the antigen binding region of the antibody or antigen binding fragment thereof that includes a VH region including an amino acid sequence having at least about 80% identity to SEQ ID NO:33 and a VL region that includes an amino acid sequence having at least about 80%, identity to SEQ ID NO:34 has a CDR-L1 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:38, a CDR-L2 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:39, and a CDR-L3 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:40.

In some aspects, the antibody or antigen binding fragment thereof includes a VH region that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:41 and a VL region that includes an amino acid sequence having at least about 80%, identity at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:42. SEQ ID NO:41 provides an amino acid sequence that includes the variable region of anti-SARS-CoV-2 antibody clone R4-21 heavy chain. SEQ ID NO: 42 provides an amino acid sequence that includes the variable region of anti-SARS-CoV-2 antibody clone R4-21 light chain.

In one aspect, the antigen binding region of the antibody or antigen binding fragment thereof that includes a VH region including an amino acid sequence having at least about 80% identity to SEQ ID NO:41 and a VL region including an amino acid sequence having at least about 80% identity to SEQ ID NO:42 has a CDR-H1 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:43, a CDR-H2 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:44, and a CDR-H3 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:45. In another aspect, the antigen binding region of the antibody or antigen binding fragment thereof that includes a VH region including an amino acid sequence having at least about 80% identity to SEQ ID NO:41 and a VL region that includes an amino acid sequence having at least about 80%, identity to SEQ ID NO:42 has a CDR-L1 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:46, a CDR-L2 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:47, and a CDR-L3 that includes an amino acid sequence having at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, at least about 99.5% identity, at least about 99.9% identity, and any number or range in between, to SEQ ID NO:48.

Antibodies or antigen binding fragments thereof provided herein further include an Fc domain. As used herein, the term Fc domain refers to an antibody region that includes at least a hinge region, a CH2 domain, and a CH3 domain, unless context clearly indicates otherwise. The terms Fc domain and Fc region may be used interchangeably unless context clearly indicates otherwise. In certain aspects, the Fc domain is an IgG domain, an IgE domain, an IgM domain, and IgD domain, an IgA domain, or an IgY domain. Fc domains of any sequence and from any species can be used, including human, ape, monkey, mouse, rabbit, goat, sheep, guinea pig, horse, and others. In certain aspects, Fc domains are engineered, i.e., non-naturally occurring or recombinant Fc domains generated using techniques of molecular biology, for example. In some aspects, the IgG domain is an IgG1 domain, an IgG2 domain, an IgG3 domain, or an IgG4 domain. In one aspect, the IgG1 domain includes an amino acid sequence of SEQ ID NO:25. In another aspect, the Fc domain is human.

Also provided herein, in some embodiments, are pharmaceutical compositions including any of the antibodies or antigen binding fragments thereof provided herein and a pharmaceutically acceptable carrier. In some aspects, the pharmaceutically acceptable carrier is conjugated to the C-terminus of one or more poly peptides of the antibody or antigen binding fragment. Any suitable means of conjugating the pharmaceutically acceptable carrier can be used, including covalent conjugation and use of linkers, for example.

In some embodiments, provided herein are isolated amino acid sequences as set forth in SEQ ID NOs:1-25 and 33-48. Also provided herein, in some embodiments, are isolated nucleic acid sequences that encode any one of the amino acid sequences of SEQ ID NOs:1-25 and 33-48.

In some embodiments, the invention further provides the expression, purification, and characterization of anti-SARS-CoV-2 antibodies, as detailed in the examples below. A signal sequence can be included in expression constructs for antibodies provided herein. Any suitable signal sequence can be used, such as a sequence of SEQ ID NO:26.

Bispecific molecules, such as bispecific antibodies (bsAbs), provide a means for simultaneously targeting multiple epitopes on the same molecular target with a single therapeutic agent. Without being limited by theory, bispecific molecules as antiviral therapeutics have the potential to confer more potent activities, lower the cost of goods, and facilitate the development of new therapeutic regimens as compared to a mixture of two monoclonal antibodies (mAbs), for example.

In an embodiment, bispecific antibodies provided herein an antigen binding region that binds to SARS-CoV-2 spike protein variants. The antigen binding region includes a VH region that includes an amino acid sequence having at least 80% identity, at least 85% identity, at least 90% identity, at least 91% identity, at least 92% identity, at least 93% identity, at least 94% identity, at least 95% identity, at least 96% identity, at least 97% identity, at least 98% identity, at least 99% identity, at least 99.5% identity, at least 99.9% identity, and any number or range in between, to a sequence selected from SEQ ID NO:1, SEQ ID NO:9, SEQ ID NO: 17, SEQ ID NO: 30, SEQ ID NO: 32, SEQ ID NO: 33, SEQ ID NO: 41 and SEQ ID NO: 53, and a VL region that includes an amino acid sequence having at least 80% identity, at least 85% identity, at least 90% identity, at least 91% identity, at least 92% identity, at least 93% identity, at least 94% identity, at least 95% identity, at least 96% identity, at least 97% identity, at least 98% identity, at least 99% identity, at least 99.5% identity, at least 99.9% identity, and any number or range in between, about 100 to 120 amino acids of an N-terminal sequence of a sequence selected from SEQ ID NO:2, SEQ ID NO:10, SEQ ID NO: 18, SEQ ID NO: 34 and SEQ ID NO:42.

Any number of amino acids of sequences as set forth in SEQ ID NO:1, SEQ ID NO:9, SEQ ID NO: 17, SEQ ID NO:30, SEQ ID NO:32, SEQ ID NO:33, SEQ ID NO:41 and SEQ ID NO: 53, that include a VH region or any number of amino acids of sequences as set forth in SEQ ID NO:2, SEQ ID NO:10, SEQ ID NO:18, SEQ ID NO:34 and SEQ ID NO:42 that include a VL region can be included in bispecific antibodies. N-terminal or C-terminal sequences of sequences provided herein having a VH region or a VL region can be included in bispecific antibodies. In one aspect, about 100 to 105 amino acids, about 100 to 110 amino acids, about 100 to 115 amino acids, about 100 to 120 amino acids, about 100 to 125 amino acids, and any number or range in between, of an N-terminal or of a C-terminal sequence of SEQ ID NO:1, SEQ ID NO:2, SEQ ID NO:9, SEQ ID NO:10, SEQ ID NO:17, SEQ ID NO:18, SEQ ID NO:30, SEQ ID NO:32, SEQ ID NO:33, SEQ ID NO:34, SEQ ID NO:41, SEQ ID NO:42 and SEQ ID NO:53, is included in bispecific antibodies provided herein. In another aspect, bispecific antibodies include a sequence of SEQ ID NO: 1, SEQ ID NO:9, SEQ ID NO:17, SEQ ID NO:30, SEQ ID NO:32, SEQ ID NO:33, SEQ ID NO:41 and SEQ ID NO:53. In yet another aspect, bispecific antibodies include about 110 amino acids of an N-terminal sequence of SEQ ID NO:2, or SEQ ID NO:10, SEQ ID NO:18, SEQ ID NO:34 or SEQ ID NO:42.

In some aspects, the bispecific antibody includes a first antigen binding region that binds to a first SARS-CoV-2 spike protein antigen and a second antigen binding region that binds to a second SARS-CoV-2 spike protein antigen. In one aspect, the bispecific antibody has the amino acid sequence as set forth in SEQ ID NO:29, 31, 49, 50, 51 or 52, or a sequence having 90% identity thereto and the binding specificity thereof. In one aspect, bispecific antibodies provided herein include an Fc domain. In another aspect, the Fc domain is an IgG domain, an IgE domain, an IgM domain, and IgD domain, an IgA domain, or an IgY domain. In yet another aspect, the Fc domain is an IgG domain. In a further aspect, the IgG domain is an IgG1 domain, an IgG2 domain, an IgG3 domain, or an IgG4 domain.

In one aspect, the scFv is linked to the C-terminus of the Fc domain. In another aspect, bispecific antibodies provided herein include a linker between the Fab domain and the scFv domain. In a further aspect, the Fab fragment is linked to the N-terminus of the Fc domain.

In an embodiment, the present disclosure provides a pharmaceutical composition that includes any one of the antibodies or an antigen binding fragment thereof provided herein or any one of the bispecific antibodies provided herein.

As used herein, “pharmaceutical composition” refers to a formulation comprising an active ingredient, and optionally a pharmaceutically acceptable carrier, diluent, or excipient. The term “active ingredient” can interchangeably refer to an “effective ingredient” and is meant to refer to any agent that is capable of inducing a sought-after effect upon administration. In one embodiment, the active ingredient includes a biologically active molecule. As used herein, the phrase “biologically active molecule” refers to a molecule that has a biological effect in a cell. In certain embodiments the active molecule may be an inorganic molecule, an organic molecule, a small organic molecule, a drug compound, a peptide, a polypeptide, such as an enzyme or transcription factor, an antibody, an antibody fragment, a peptidomimetic, a lipid, a nucleic acid such as a DNA or RNA molecule, a ribozyme, hairpin RNA, siRNA (small interfering RNAs) of varying chemistries, miRNA, siRNA-protein conjugate, an siRNA-peptide conjugate, and siRNA-antibody conjugate, an antagomir, a PNA (peptide nucleic acid), an LNA (locked nucleic acids), or a morpholino. In certain illustrative embodiments, the active agent is a polypeptide or peptide.

By “pharmaceutically acceptable” it is meant the carrier, diluent or excipient must be compatible with the other ingredients of the formulation and not deleterious to the recipient thereof, nor to the activity of the active ingredient of the formulation. Pharmaceutically acceptable carriers, excipients or stabilizers are well known in the art, for example Remington's Pharmaceutical Sciences, 16^{th} edition, Osol, A. Ed. (1980). Pharmaceutically acceptable carriers, excipients, or stabilizers are nontoxic to recipients at the dosages and concentrations employed, and may include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (for example, Zn-protein complexes); and/or non-ionic surfactants such as TWEEN™, PLURONICS™ or polyethylene glycol (PEG). Examples of carrier include, but are not limited to, liposome, nanoparticles, ointment, micelles, microsphere, microparticle, cream, emulsion, and gel. Examples of excipient include, but are not limited to, anti-adherents such as magnesium stearate, binders such as saccharides and their derivatives (sucrose, lactose, starches, cellulose, sugar alcohols and the like) protein like gelatin and synthetic polymers, lubricants such as talc and silica, and preservatives such as antioxidants, vitamin A, vitamin E, vitamin C, retinyl palmitate, selenium, cysteine, methionine, citric acid, sodium sulfate and parabens. Examples of diluent include, but are not limited to, water, alcohol, saline solution, glycol, mineral oil, and dimethyl sulfoxide (DMSO).

The term “pharmaceutically acceptable salt” refers to physiologically and pharmaceutically acceptable salts of the compounds of the invention, e.g., salts that retain the desired biological activity of the parent compound and do not impart undesired toxicological effects thereto.

The pharmaceutical composition may also contain other therapeutic agents, and may be formulated, for example, by employing conventional vehicles or diluents, as well as pharmaceutical additives of a type appropriate to the mode of desired administration (for example, excipients, preservatives, etc.) according to techniques known in the art of pharmaceutical formulation.

In one aspect, antibodies or antigen binding fragments or bispecific antibodies of the pharmaceutical compositions provided herein include a pharmaceutically acceptable carrier conjugated to the C-terminus of one or more polypeptides of the antibody or antigen binding fragment thereof. In another aspect, pharmaceutical compositions provided herein include a bispecific antibody.

In one embodiment, the invention provides an isolated amino acid sequence as set forth in SEQ ID NOs:1-53. In one embodiment, the present disclosure also provides an isolated nucleic acid sequence encoding the antibody, an antigen-binding fragment thereof, or the bispecific antibody of the invention. In another embodiment, the invention provides an isolated nucleic acid encoding any one of SEQ ID NOs:1-53.

As used herein, the term “nucleic acid” refers to polynucleotides such as deoxyribonucleic acid (DNA) or ribonucleic acid (RNA). Nucleic acids include but are not limited to genomic DNA, cDNA, mRNA, iRNA, miRNA, tRNA, ncRNA, rRNA, and recombinantly produced and chemically synthesized molecules such as aptamers, plasmids, anti-sense DNA strands, shRNA, ribozymes, nucleic acids conjugated and oligonucleotides. According to the invention, a nucleic acid may be present as a single-stranded or double-stranded and linear or covalently circularly closed molecule. A nucleic acid can be isolated. The term “isolated nucleic acid” means, that the nucleic acid (i) was amplified in vitro, for example via polymerase chain reaction (PCR), (ii) was produced recombinantly by cloning, (iii) was purified, for example, by cleavage and separation by gel electrophoresis, or (iv) was synthesized, for example, by chemical synthesis. A nucleic can be employed for introduction into, i.e., transfection of, cells, in particular, in the form of RNA which can be prepared by in vitro transcription from a DNA template. The RNA can moreover be modified before application by stabilizing sequences, capping, and polyadenylation.

The terms “peptide,” “polypeptide” and “protein” are used interchangeably herein and refer to any chain of at least two amino acids, linked by a covalent chemical bound. As used herein polypeptide can refer to the complete amino acid sequence coding for an entire protein or to a portion thereof. A “protein coding sequence” or a sequence that “encodes” a particular polypeptide or peptide, is a nucleic acid sequence that is transcribed (in the case of DNA) and is translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the coding sequence.

Presented below are examples discussing the antibodies and bispecific antibodies of the invention, contemplated for the discussed applications. The following examples are provided to further illustrate the embodiments of the present invention but are not intended to limit the scope of the invention. While they are typical of those that might be used, other procedures, methodologies, or techniques known to those skilled in the art may alternatively be used.

### EXAMPLES

### Example 1

### Antibody Generation Form the Omnimab Library

To generate antibodies against SARS-CoV-2 spike protein, selections with an OmniMab phagemid library were carried out. The phagemid library was built up by AP Biosciences Inc. (APBio Inc.) from a collection of peripheral blood mononuclear cells from over a hundred healthy donors.

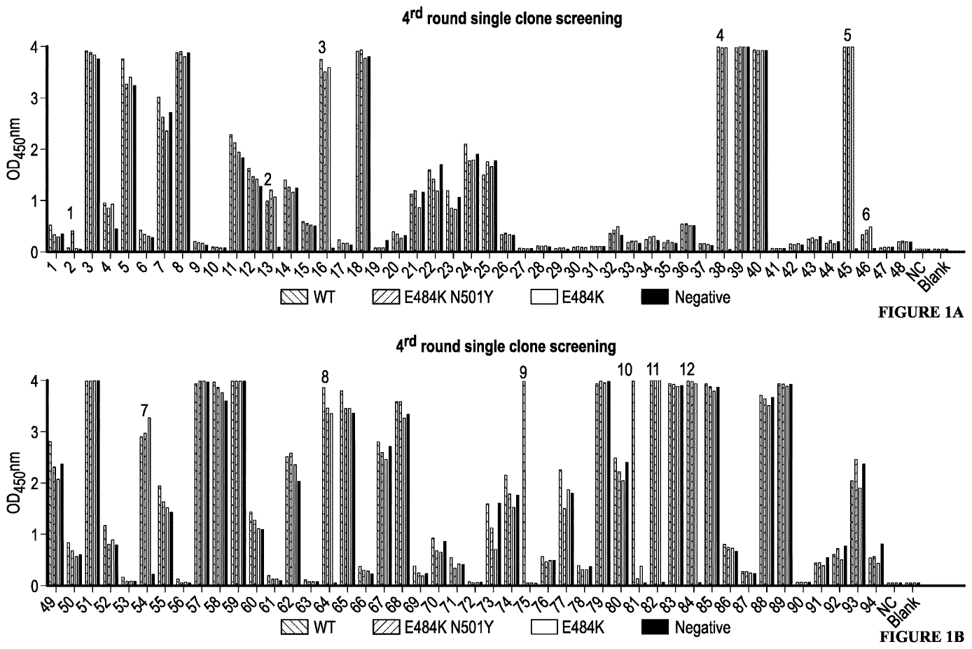

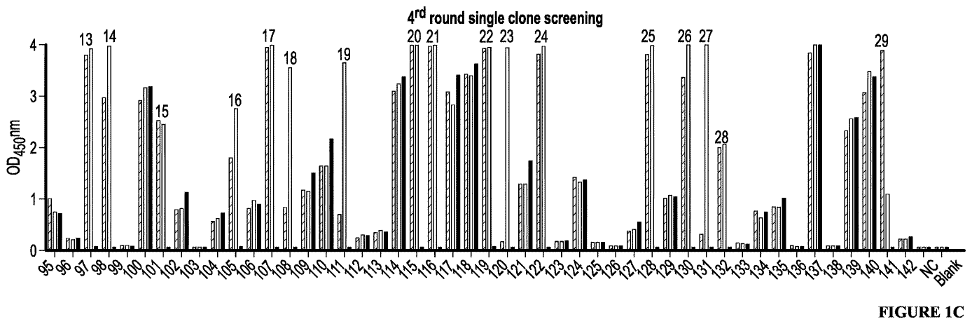

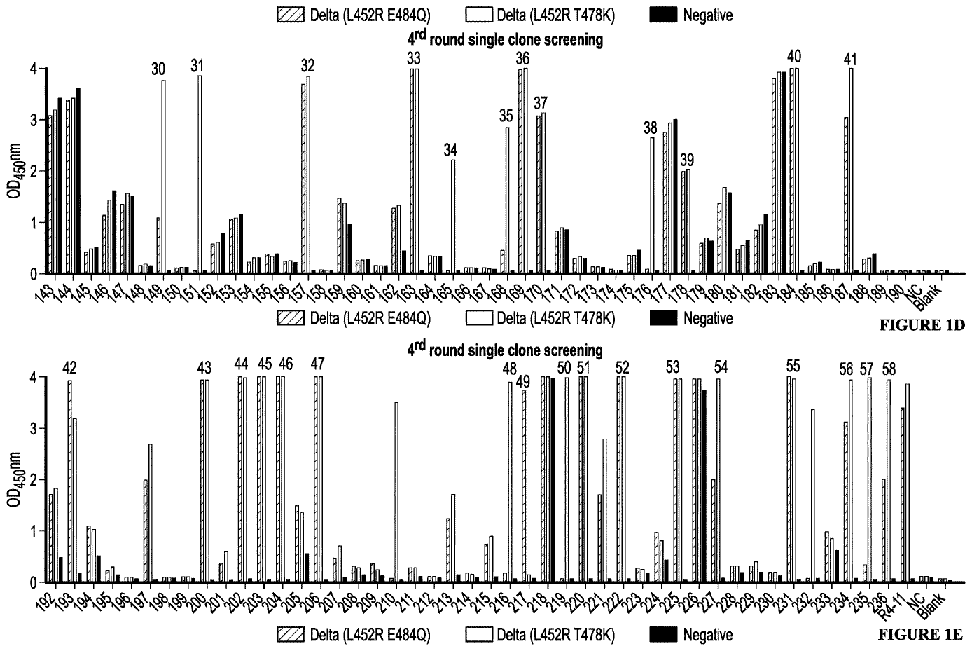

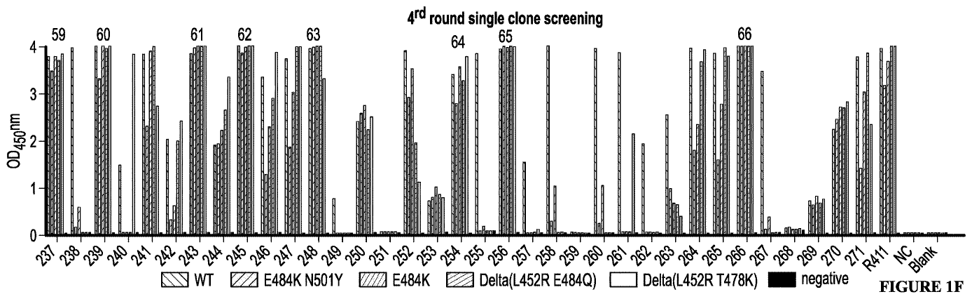

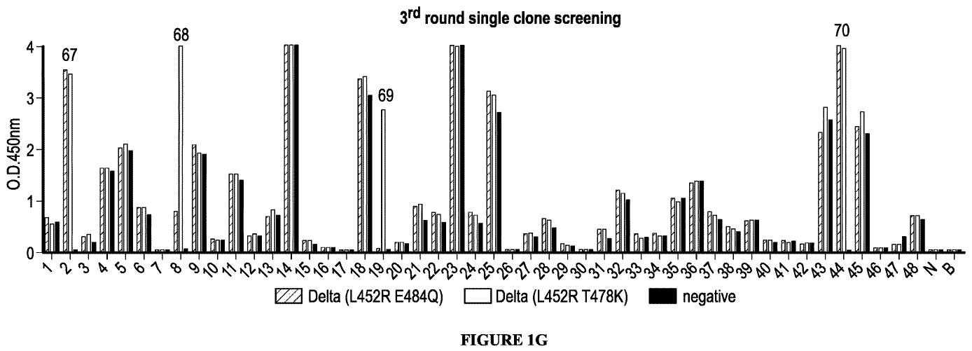

SARS-CoV-2 spike protein specific binders were screened and isolated from the OmniMab phage-display library using Hyperphage (M13K07ΔρIII, Progen, Heidelberg, Germany). Solid phase panning was performed using recombinant SARS-CoV-2 spike RBD His-tag proteins (R&D; #10500-CV, #10788-CV, #10747-CV, #10846-CV, #10876-CV). After three rounds of selection, the specific binders of spike RBD of known SARS-CoV-2 variants were screened by direct ELISA (FIGS. 1A-1G). The binders with specific binding to spike RBD were selected and numbered (FIGS. 1A to 1G). Selected binders were sent for sequencing to confirm the integrity and diversity of the heavy chains and light chains. The nomenclature for all the clones used in FIGS. 1A-1G is listed in FIG. 1H.

### Example 2

### Subcloning, Expression, and Purification of SARS-CoV-2 Specific Binding Proteins in the Form of IgGs

The sequence of heavy chains and light chains of positive SARS-CoV-2 spike RBD binders were amplified, digested and subcloned into an IgG expression vector (AP Biosciences) that carrying the IgG1 constant region (SEQ ID NO:17). After sequence validation, plasmids containing binder sequences were prepared and transfected into ExpiCHO cells (Invitrogen) for antibody production. After 6 days of culture, antibody secreted into serum-free medium was affinity purified from culture supernatant by Protein A chromatography. Purified antibody was concentrated, followed by dialysis in PBS buffer. The antibody concentration was determined using a NanoDrop2000 spectrophotometer. The antibody purity and integrity were analyzed by NuPAGE (ThermoFisher, Cat. No. NP0321BOX) under non-reducing or reducing conditions. Antibody integrity was visualized by Coomassie Brilliant Blue G-250 staining.

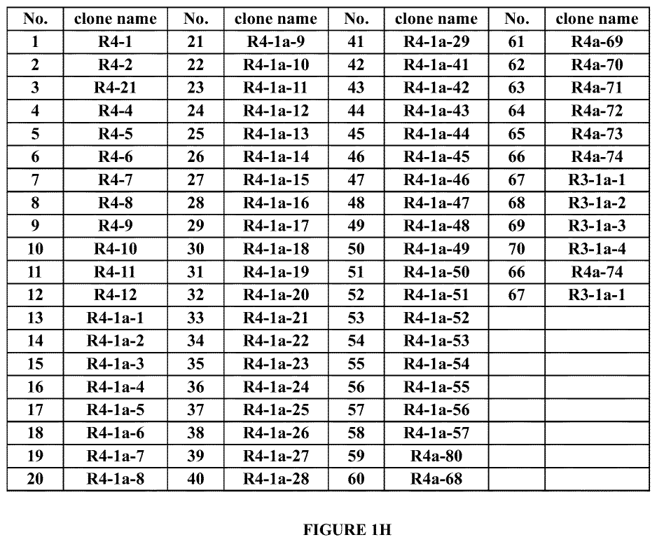

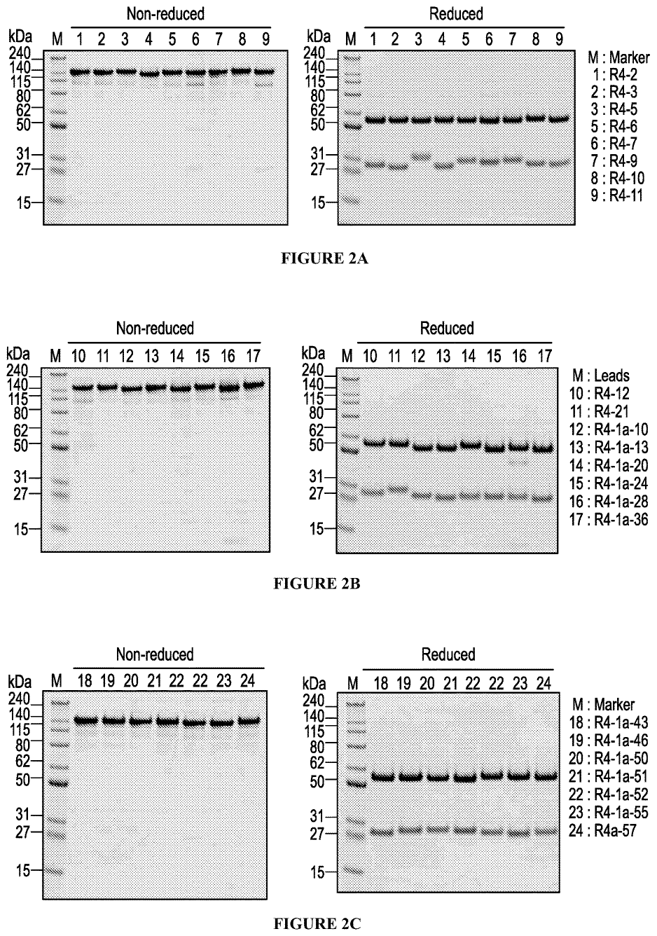

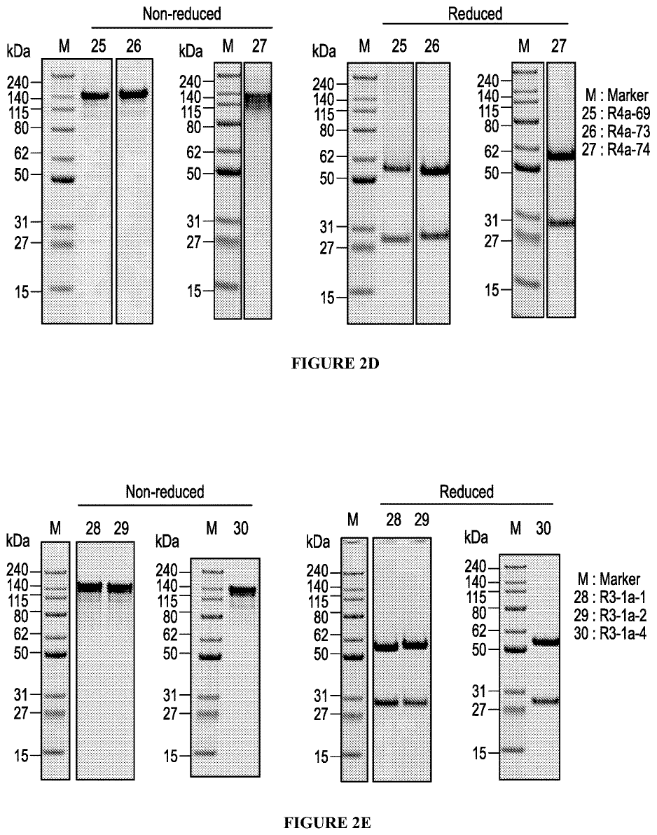

FIGS. 2A-2E show representative PAGE gel analysis of different batches of purified anti-SARS-CoV-2 antibody leads. Results indicated that proteins had a molecular weight of about 145 kDa under non-reducing conditions, and heavy chain and light chain had a molecular weight of 55 kDa and 25 kDa, respectively, under reducing conditions. More than 85% purity could be obtained by one step of Protein A chromatography. Some clones of phage binders were abandoned after converting to the IgG format due to the none or low productivity in CHO cells.

### Example 3

### Binding of Anti-SARS-CoV-2 Monoclonal Antibody to SARS-CoV-2 Spike Protein with Known Variants

To perform a direct ligand binding assay of anti-SARS-CoV-2 antibody, recombinant SARS-CoV-2 spike protein with known mutations (Table 2) were pre-coated on the wells of Nunc-Immuno Maxisorp 96 well plates and incubated at 4° C. overnight. The SARS-CoV-2 recombinant spike protein solution was removed, and the plates were washed three times with 0.4 ml of wash buffer (0.1% Tween-20 in PBS). 0.4 ml blocking buffer (5% low-fat milk powder in PBS) was added to all wells and incubated at room temperature for 1 hour. The blocking buffer was removed and washed three times with 0.4 ml wash buffer. Pre-coated wells were incubated with serial dilutions of purified anti-SARS-CoV-2 antibodies in 0.1 ml PBS for 1 hour at room temperature. The antibody solution was removed, and the plates were washed three times with 0.4 ml wash buffer. HRP-conjugated goat anti-human IgG, F(ab′)2 specific F(ab′)2 antibody (Jackson Immunoresearch #109-036-097) was diluted 1:2000 with PBS and added at 0.1 ml per well. The plates were incubated for 1 hour at room temperature and washed three times with 0.4 ml wash buffer per well. HRP substrate TMB (Invitrogen) 0.1 ml were added into wells and incubated for 1 to 5 minutes at room temperature. 0.05 ml 1N HCl was added to stop the reaction and absorbances were read at 450 nm on a Bio-Tek Spectra. The EC50 of anti-SARS-CoV-2 antibody binding to SARS-CoV-2 spike protein was calculated using nonlinear regression of Prism software (GraphPad, San Diego, CA).

TABLE 2
Recombinant SARS-CoV-2 spike protein with known mutations.
SARS-CoV-2 spike protein

| Strain | Description | Catalog number |
|---|---|---|
| Wild type | SARS-CoV-2 Spike Protein | R&D, Cat. #10549-CV |
| Alpha Variant | SARS-CoV-2 +E484K B.1.1.7 Spike Protein | R&D, Cat. #10824-CV |
| Lambda Variant | SARS-CoV-2 C.37 Spike His Protein | R&D, Cat. #10887-CV |
| Delta Variant (India) | SARS-CoV-2 B.1.617.2 Spike Protein | R&D, Cat. #10878-CV |
| Delta Plus Variant | SARS-CoV-2 AY.1 Spike Protein | R&D, Cat. #10922-CV |
| Omicron | SARS-CoV-2 B.1.1.529 Spike Protein | AcroBiosystems, Cat. # |
| | | SPN-C52Hz |
| Kappa Variant L452R | Recombinant SARS-CoV-2 B.1.617.1 Spike | R&D, 10846-CV |
| E484Q | RBD His Protein, CF | |
| Delta Variant (India) | Recombinant SARS-CoV-2 B.1.617.2 Spike | R&D, 10876-CV |
| L452R T478K | RBD His Protein, CF | |
| Omicron B.1.1.529 | Recombinant SARS-CoV-2 B.1.1.529 Spike | R&D, 11060-CV |
| | His-tag Protein, CF | |
| Omicron BA.2 | Recombinant SARS-CoV-2 BA.2 Spike | R&D, 11109-CV |
| | (GCN4-IZ) His Protein, CF | |
| Omicron BA.4 | SARS-CoV-2 Spike Trimer, His Tag | AcroBiosystems, SPN- |
| | (BA.4/Omicron) (MALS verified) | C5229 |
| Omicron BA.5 | SARS-CoV-2 Spike Trimer, His Tag | AcroBiosystems, SPN- |
| | (BA.5/Omicron) (MALS verified) | C522e |

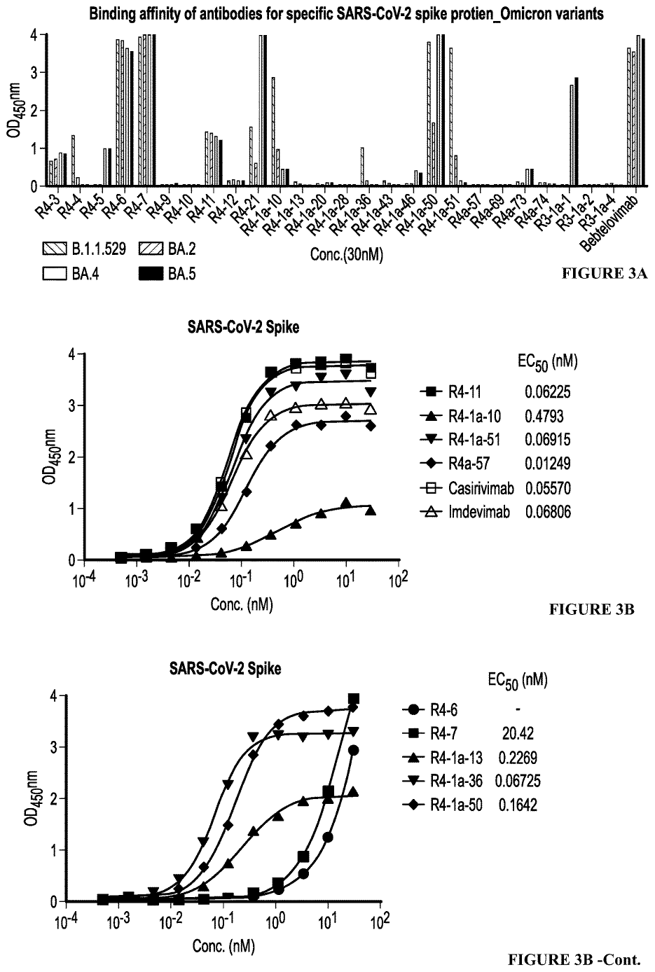

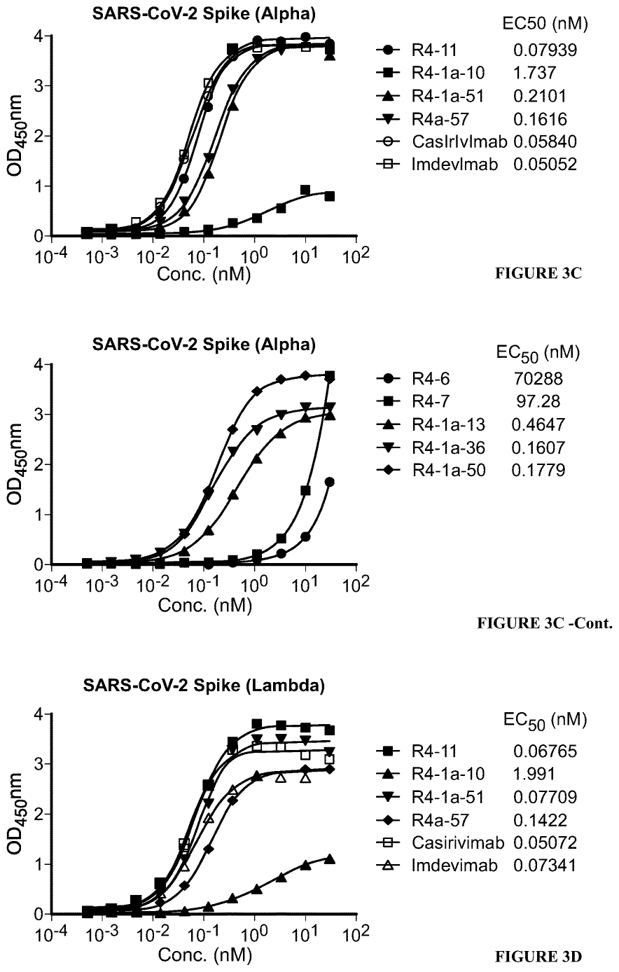

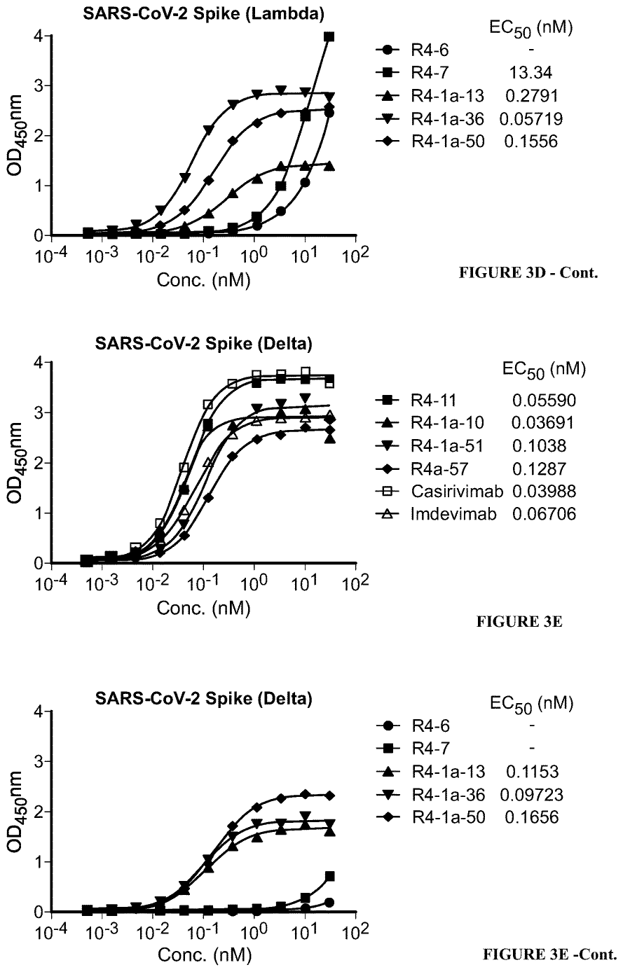

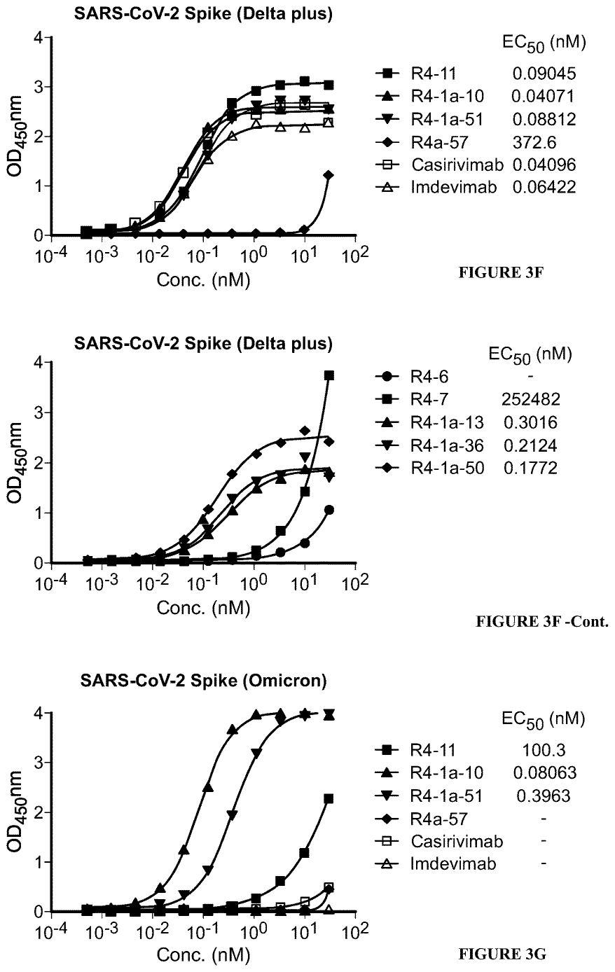

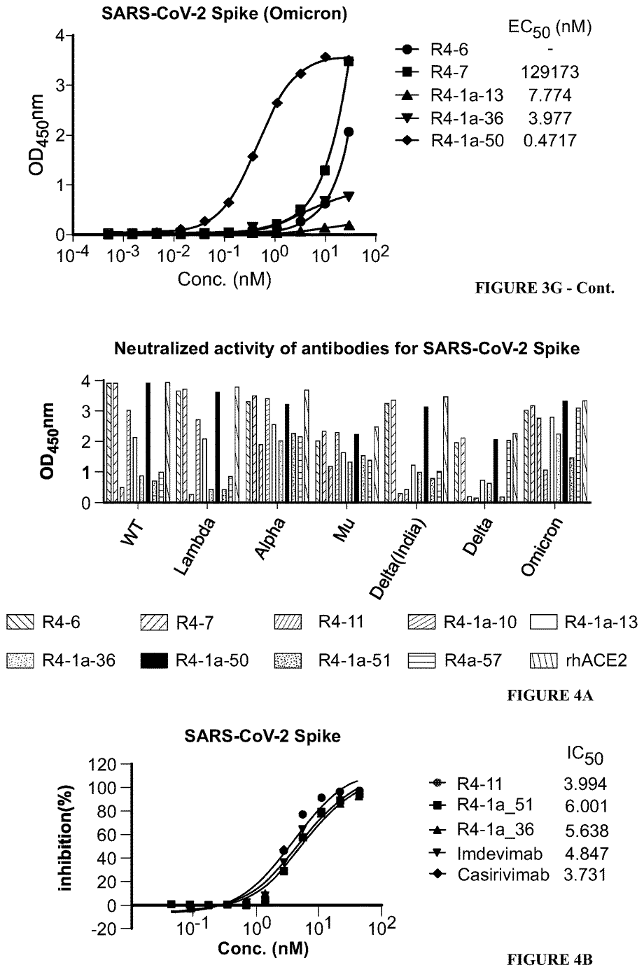

FIG. 3A shows the binding activities of antibody leads to a fixed concentration (30 nM) of spike protein of Omicron and subvariants. Among the screened leads, R3-1a-1 and R4-21 showed the better binding activity in BA.4 and BA.5 strains especially. R4-1a-10, and R4-1a-51 showed the better binding activity in Omicron strain (B1.1.529). The mAbs were selected for construction to bispecific antibodies.

Especially, clone #R4-11 showed the most remarkable binding to spike proteins of WT, Alpha, Lambda, Delta, and Delta plus (FIGS. 3B-3F). Instead, clone #R4-1a-10 and #R4-1a-51 showed the best binding activity to spike proteins of Omicron and Omicron BA.2. (FIG. 3G).

### Example 4

### Anti-SARS-CoV-2 Monoclonal Antibody Inhibit Binding of SARS-CoV-2 Spike Protein to ACE-2

To determine the neutralizing activities of antibody leads to inhibit the binding of SARS-CoV-2 spike protein to its receptor ACE-2, the competitive ELISA assay was conducted. Briefly, purified anti-SARS-CoV-2 antibodies were dialyzed in Dulbecco's Phosphate buffered saline (DPBS) and ACE2 were conjugated with biotin (abeam #ab201795). Nunc-Immuno Maxisorp 96 well plates were pre-coated with recombinant SARS-CoV-2 spike protein and incubated at 4° C. overnight. Coated wells were blocked with blocking buffer (DPBS containing 1% BSA and 0.1% Tween-20) at room temperature for 1 hour. After washing three times with 0.35 mL wash buffer (DPBS containing 0.1% Tween-20), serial 2-fold dilutions of anti-SARS-CoV-2 antibody (from 30 to 0 nM) and a fixed concentration (5.88 nM) of biotin-conjugated ACE2 in blocking buffer were added and incubated at room temperature for 1 hour. The antibody-ACE2 mixtures was removed and washed three times with 0.35 mL wash buffer. The HRP-Streptavidin solution was added and incubated at room temperature for 1 hour. After washing three times with 0.35 mL of wash buffer, 0.1 mL TMB solution was added and incubate for 5 minutes until color development. The reaction was stopped by 0.05 mL 1N HCl and read Absorbance 450 nm using SpectraMax iD3 microplate reader. The percentage of neutralization was calculated by the following formula: Neutralization (%)=(OD450 nm of ACE2 alone−OD450 nm of antibody lead+ACE2)/OD450 nm of ACE2 alone)×100%. IC50 was calculated by nonlinear regression using GraphPad Prism software.

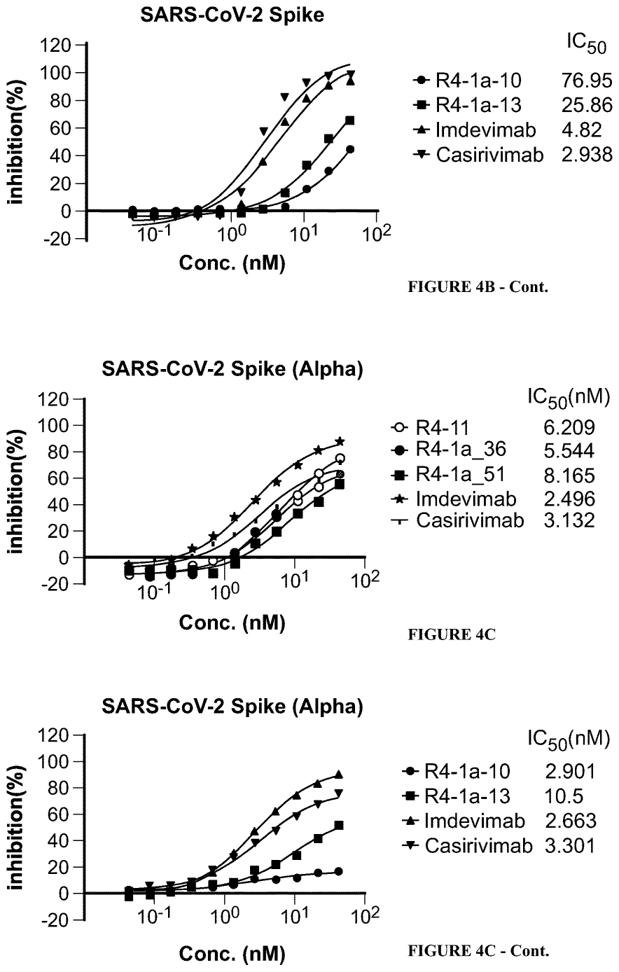

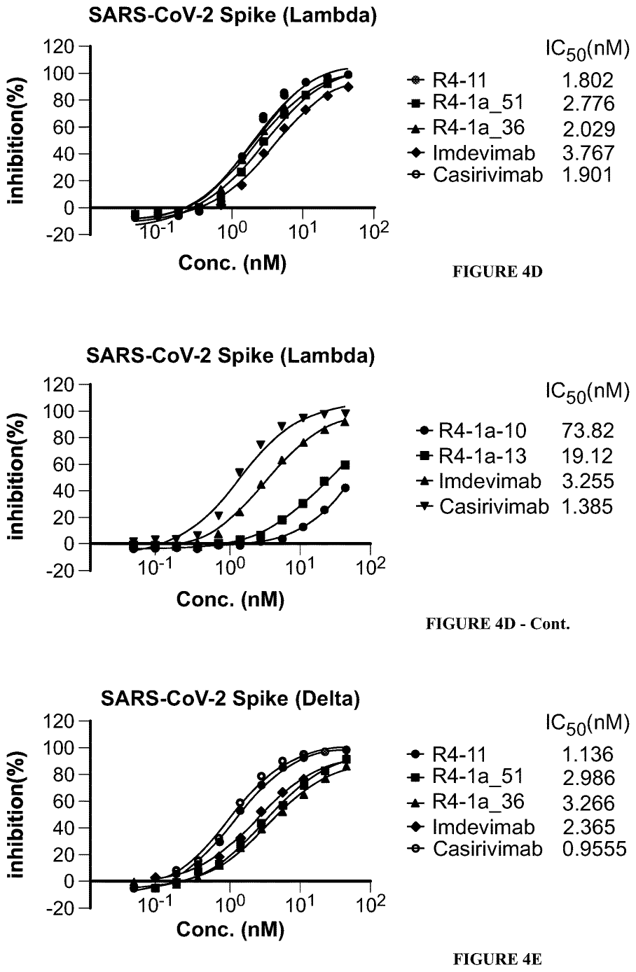

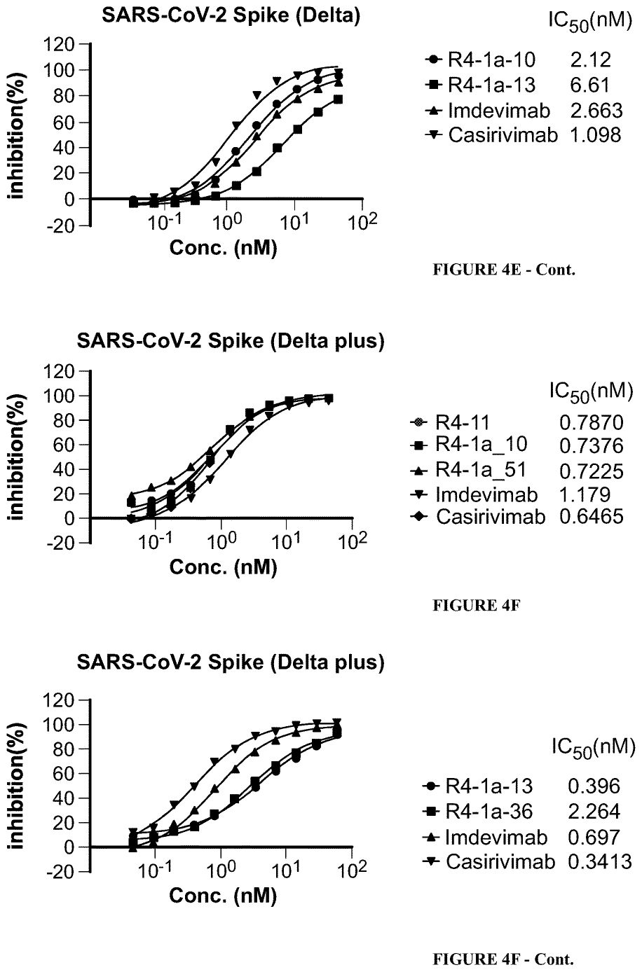

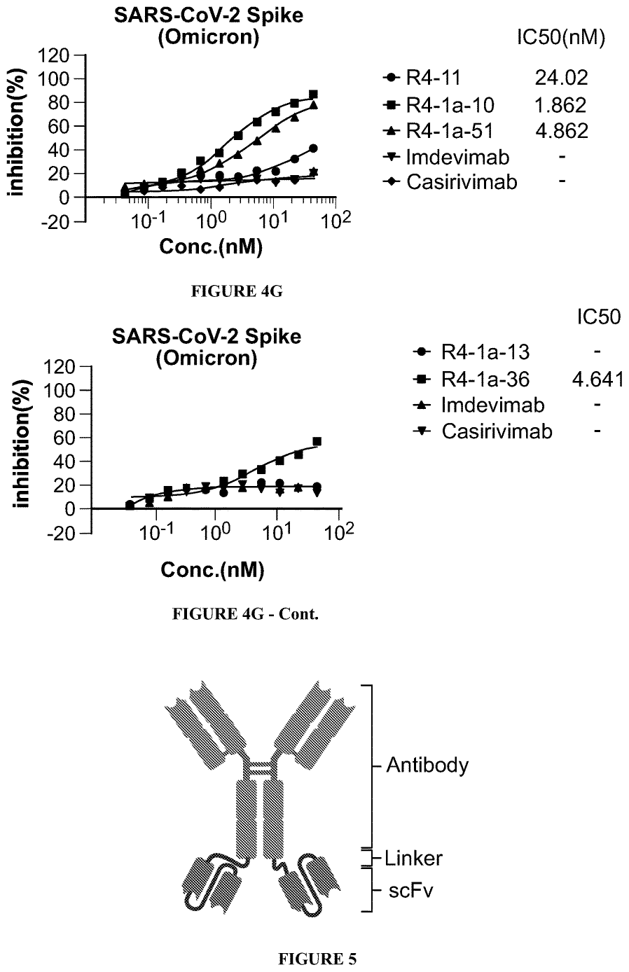

FIGS. 4A-4G show the neutralizing activities of anti-SARS-CoV-2 antibody leads using spike proteins of SARS-CoV-2 variants. Antibody leads were first screen for neutralizing activity at a fixed concentration. Corresponding to the binding activity observed in FIGS. 3A-3G, clone #R4-11 showed the most potent and broad neutralization against spike proteins of WT, Lambda, Delta, and Delta plus. However, clone #R4-11, #R4-1a-10, #R4-1a-13, #R4-1a-36, and #R4-1a-51 showed obvious neutralizing activities to spike protein variants, and the IC50 values were further determined by serial antibody dilutions. Calculated IC50 values for anti-SARS-CoV-2 specific antibody clone R4-1a-10 and R4-1a-50 showed good neutralizing activity in omicron strain.

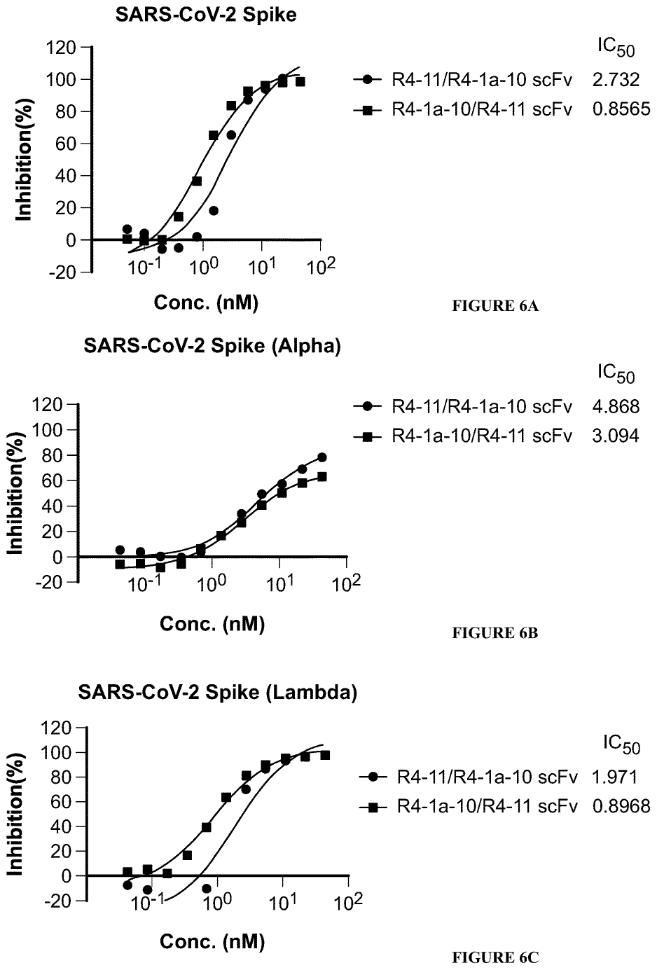

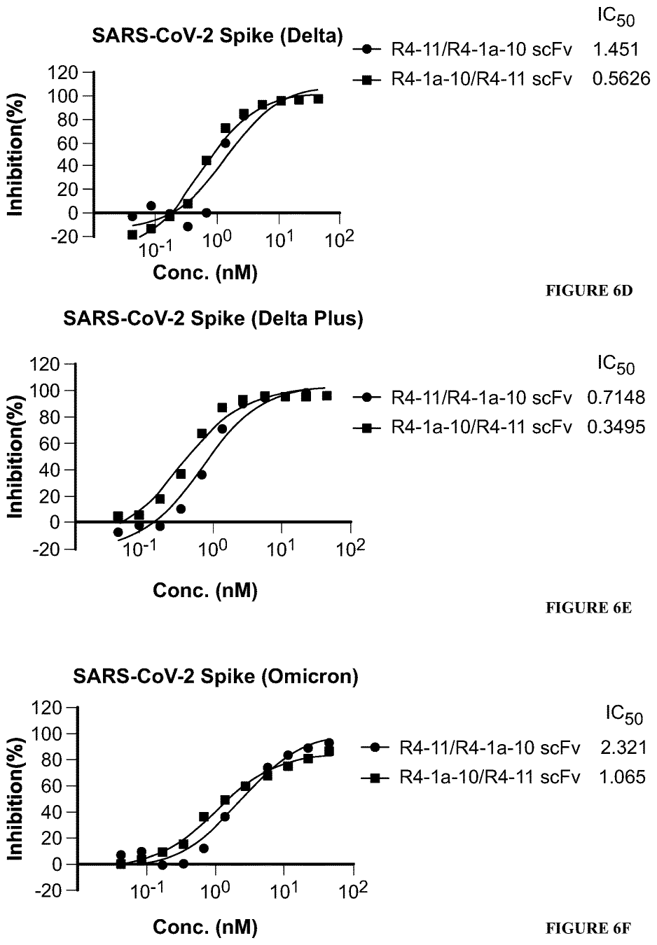

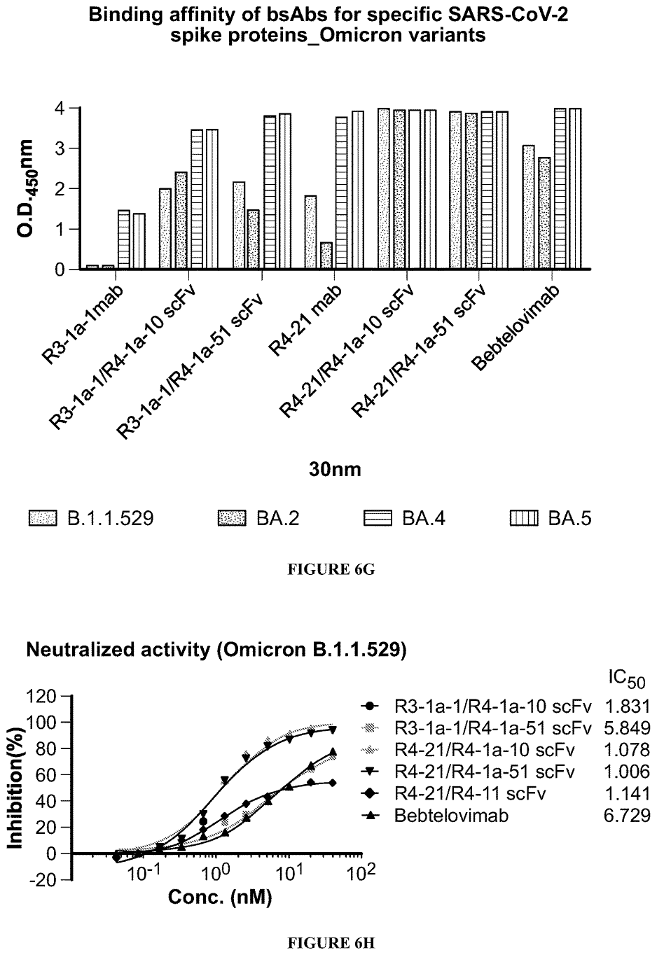

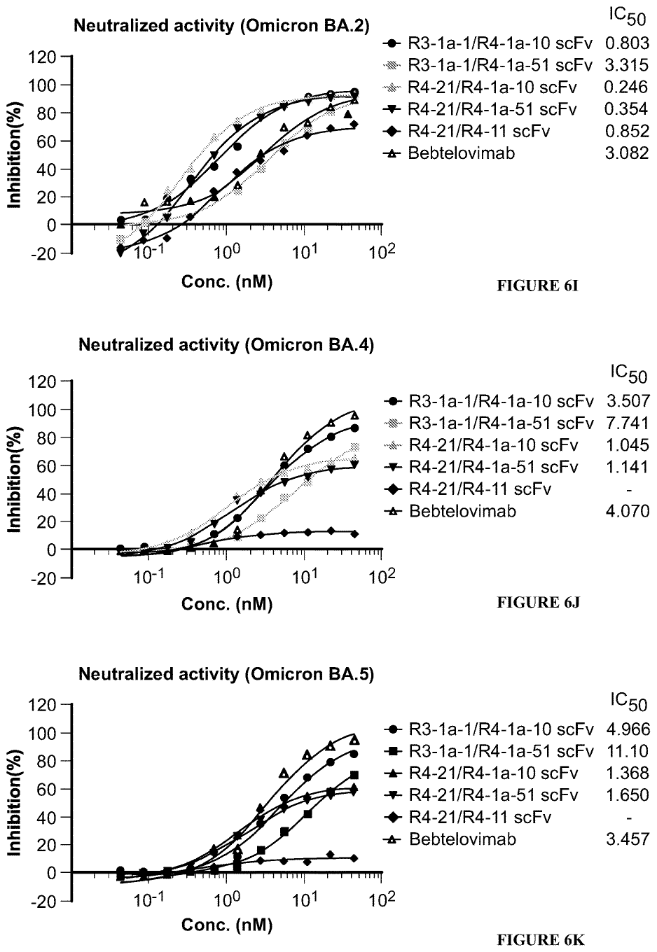

The results in FIG. 6H-6K, using the calculated IC50 value (defined as the concentration of antibody required to reduce the binding of the viral spike protein to ACE2 by 50%) as an indicator of blocking potency, show the neutralizing activities of anti-SARS-CoV-2 bispecific antibodies. The candidate R4-21/R4-1a-10 scFv and R3-1a-1/R4-1a-10 scFv had the good ability to block the binding of Omicron, BA.2, BA.4 and BA.5 spike protein to the isolated ACE2 Protein.

### Example 5

### Anti-SARS-CoV-2 Monoclonal Antibody Binding Affinity on Recombinant Spike Protein Variants Measured by Fortebio

The affinity of the AP711 mAbs was determined by FortéBio® (Menlo Park, Calif.) biosensor analysis. In this method, the antibodies were loaded on AHC (Anti-Human IgG Fc Capture) biosensor (Cat #18-5060) at 5 μg/mL for signal reach to approximate 0.5 nm. Sensors then were exposed to recombinant COVID spike proteins solutions with 2 times serial dilutions for 8 points. Measurement was performed as follows: 1 min baseline in kinetics buffer (0.2% bovine serum albumin (BSA) in 1×DPBS diluted from 10×DPBS (Invitrogen, Cat. #14200-075) with ddH2O, 0.05% Tween-20), 5 minutes association in 8 different recombinant COVID spike proteins solutions and 5 minutes dissociation into kinetics buffer. The kinetic parameters were calculated and produced by Octet Data Acquisition and Analysis Software.

The binding affinity of anti-SARS-CoV-2 mAbs against different recombinant COVID spike proteins were assessed by biolayer interferometry (BLI), and the results were summarized in Table 3. In summary, all mAbs exhibited nM affinities of COVID Omicron variants except R4-1a-51 and R3-1a-1. R4-1a-51 against BA.4 variant of 1.03E-08 M and BA.5 variant of 1.25E-08. R3-1a-1 against Omicron cannot be detected, BA.4 and BA.5 variants of 1.03E-08 and 1.25E-08 separately.

TABLE 3
Summary of anti-SARS-CoV-2 mAbs binding affinities against COVID spike proteins.

| | KD (M) | | | | | | | | |
|---|---|---|---|---|---|---|---|---|---|
| Sample | WT | Alpha | Lambda | Delta | Delta plus | omicron | BA.2 | BA.4 | BA.5 |
| R4-11 | 4.05E−09 | 5.66E−09 | 1.23E−09 | 1.53E−09 | 2.29E−09 | 7.93E−09 | N.D. | N.D. | N.D. |
| R4-1a-10 | 1.87E−09 | 5.57E−09 | 1.01E−09 | 2.34E−09 | 1.79E−09 | 5.71E−10 | 3.23E−09 | 6.86E−09 | 7.86E−09 |
| R4-1a-51 | 2.72E−09 | 1.21E−08 | 1.21E−09 | 4.13E−09 | 1.26E−09 | 3.49E−09 | 1.03E−08 | 1.11E−05 | 2.53E−04 |
| R3-1a-1 | N.D. | N.D. | N.D. | N.D. | N.D. | N.D. | 4.36E−09 | 1.03E−08 | 1.25E−08 |
| R4-21 | N.D. | N.D. | N.D. | N.D. | N.D. | 2.58E−09 | 2.32E−09 | 1.62E−09 | 2.63E−09 |
| Imdevimab | 2.32E−09 | 8.31E−10 | 5.63E−10 | 1.53E−09 | 3.05E−10 | 4.56E−07 | N.D. | N.D. | N.D. |
| Casirivimab | 2.85E−09 | 3.40E−09 | 5.11E−10 | 4.84E−10 | 1.08E−09 | 7.82E−09 | N.D. | N.D. | N.D. |

### Example 6

### Construction, Expression and Purification of Anti-SARS-CoV-2 Bispecific Antibodies

Bispecific antibodies having the general structure illustrated in FIG. 5 were generated.

Anti-SARS-CoV-2 antibody clone R3-1a-1 was used in IgG1, and clone R4-1a-10 and R4-1a-51 were used in scFv format and fused to the C-terminal of the anti-SARS-CoV-2 clone R4-3a-1 antibody Fc region separately (R3-1a-1/R4-1a-10 scFv; R3-1a-1/R4-1a-51 scFv). On the other hand, Anti-SARS-CoV-2 antibody clone R4-21 was used in IgG1, and clone R4-1a-10 and R4-1a-51 were used in scFv format and fused to the C-terminal of the anti-SARS-CoV-2 clone R4-3a-1 antibody Fc region separately (R4-21/R4-1a-10 scFv; R4-21/R4-1a-51 scFv). The amino acid sequences of heavy chain are shown in SEQ ID NO:65 to SEQ ID NO:68.

Bispecific antibody constructs that include an anti-SARS-CoV-2 antibody Fc region fused

with scFv are shown in Table 1 below (Sequence), with a schematic shown in FIG. 5. A short flexible peptide (G4S)4 linker (SEQ ID NO:28) was placed between R3-1a-1 or R4-21 C-terminal of Fc region and N-terminal of R4-1a-10 scFv or R4-1a-51 scFv to ensure correct folding and minimize steric hindrance. The amino acid sequences of anti-SARS-CoV-2 heavy chain are shown in SEQ ID NO:29. Antibody Fc fusion protein constructs were expressed using the Gibco ExpiCHO Expression System and purified from the cell culture supernatant of transfected cells via 1-step Protein G chromatography.

TABLE 1
Sequences of Defined CDR Regions of Monoclonal Antibodies and Single
Chain Variable Fragments (scFv) of Bispecific Antibodies.

| | Sequence | | |
|---|---|---|---|
| Name | CDR1 | CDR2 | CDR3 |
| Anti-SARS-CoV-2#R4-11-H | GGAFSSY | IPILGI | GSGYYY |
| | (SEQ ID NO: 3) | (SEQ ID NO: 4) | (SEQ ID NO: 5) |
| Anti-SARS-CoV-2#R4-11-L | SGSSSNIANNAVN | YDDLLPS | AAWDDSLNGPV |
| | (SEQ ID NO: 6) | (SEQ ID NO: 7) | (SEQ ID NO: 8) |
| Anti-SARS-CoV-2#R4-1a-10-H | GGSFSGN | NHSGS | GLLDY |
| | (SEQ ID NO: 11) | (SEQ ID NO: 12) | (SEQ ID NO: 13) |
| Anti-SARS-CoV-2#R4-1a-10-L | SGSDSNIGNNPVN | YDNFLAS | ASWDDSLNGVV |
| | (SEQ ID NO: 14) | (SEQ ID NO: 15) | (SEQ ID NO: 16) |
| Anti-SARS-CoV-2#R4-1a-51-H | GYTFTGY | NPNSGG | GYMDY |
| | (SEQ ID NO: 19) | (SEQ ID NO: 20) | (SEQ ID NO: 21) |
| Anti-SARS-CoV-2#R4-1a-51-L | SGSSSNIGSSTVN | SNHQRPS | AAWDDSLNGVV |
| | (SEQ ID NO: 22) | (SEQ ID NO: 23) | (SEQ ID NO: 24) |
| Anti-SARS-CoV-2#R3-1a-1-H | GGTFSSY | IPILGI | DGGGSSGYHDY |
| | (SEQ ID NO: 35) | (SEQ ID NO: 36) | (SEQ ID NO: 37) |
| Anti-SARS-CoV-2#R3-1a-1-L | TRSSGSIASKYVQ | EDDQRPS | QSYDDNTAV |
| | (SEQ ID NO: 38) | (SEQ ID NO: 39) | (SEQ ID NO: 40) |
| Anti-SARS-CoV-2#R4-21-H | GYSFTSY | YPGDSD | QTPYYDSSDYYLGDAFDI |
| | (SEQ ID NO: 43) | (SEQ ID NO: 44) | (SEQ ID NO: 45) |
| Anti-SARS-CoV-2#R4-21-L | TGTSSDVGGYNYVS | DVRNRPS | GSYTSSSTLG |
| | (SEQ ID NO: 46) | (SEQ ID NO: 47) | (SEQ ID NO: 48) |

| | |
|---|---|
| Anti-SARS-CoV-2 R4-11-scFv | QSVLTQPPSVSEAPGQRVTISCSGSSSNIANNAVNWYQQLPGQAPKLLIF |
| | YDDLLPSWVSDRFSGSKSGTSASLAISGLQSEDEADYYCAAWDDSLNG |
| | PVFGGGTKLTVLGGGGGSGGGGSGGGGSGGGGSQVQLVQSGAEVKKP |
| | GSSVKVSCKASGGAFSSYAISWVRQAPGQGLEWMGRIIPILGIANYAQK |
| | FQGRVTITADKSTSTAYMELSSLRSEDTAVYYCAGGSGYYYWGQGTTV |
| | TVSSA (SEQ ID NO: 30) |
| Anti-SARS-CoV-2 R4-1a-10- | QSVLTQPSSVSAAPWQRVTISCSGSDSNIGNNPVNWYQHVPGKAPTLLI |
| scFv | YYDNFLASKISDRFSGSKSGTSASLAISGLQSDDEADYYCASWDDSLN |
| | GVVFGGGTKLTVLGGGGSGGGGSGGGGSGGGGSQVQLQQWGAGLLK |
| | PSETLSLTCAVYGGSFSGNYWSWIRQPPGKGLEWIGEINHSGSTNYNPS |
| | LKSRVTISVDTSKNQFSLKLSSVTAADTAVYYCARGLLDYWGQGTLVT |
| | VSSA (SEQ ID NO: 32) |
| Anti-SARS-CoV-2 R4-1a-51- | QSVLTQPPSASGTPGQRVTISCSGSSSNIGSSTVNWYQQLPETAPKLLIYS |
| scFv | NHQRPSGVPDRFSGSKSGTSASLALSGLQSEDEAEYYCAAWDDSLNGV |
| | VFGGGTKLTVLGGGGSGGGGSGGGGSGGGGSQVQLVQSGAEVKKPG |
| | ASVKVSCKASGYTFTGYYMHWVRQAPGQGLEWMGRINPNSGGTNYA |
| | QKFQGRVAMTRDTSISTAYMELSRLRSDDTAVYYCARGYMDYWGQGT |
| | PVTVSSA (SEQ ID NO: 52) |

*CDR regions define by Chothia

### Example 7

### Neutralizing Activities of Bispecific Antibody Leads Analysis by Competitive ELISA Assay—Anti-SARS-CoV-2 Monoclonal Antibody Inhibit Binding of SARS-CoV-2 Spike Protein to ACE-2

The binding activities of bispecific antibody leads were studied by ELISA assay as described above in Example 3.

FIGS. 6A-6F shows IC50 of R4-1a-10/R4-11 scFv, which was found lower than that of R4-11/R4-1a-10 scFv in all SARS-CoV-2 spike variants. As can be seen in FIG. 6G the bispecific antibodies R4-21/R4-1a-10 scFv and R4-21/R4-1a-51 showed high binding activity in Omicron and subvariants.

### Example 8

### Pseudovirus Neutralizing Anti-SARS-CoV-2 Bispecific Antibody Assay

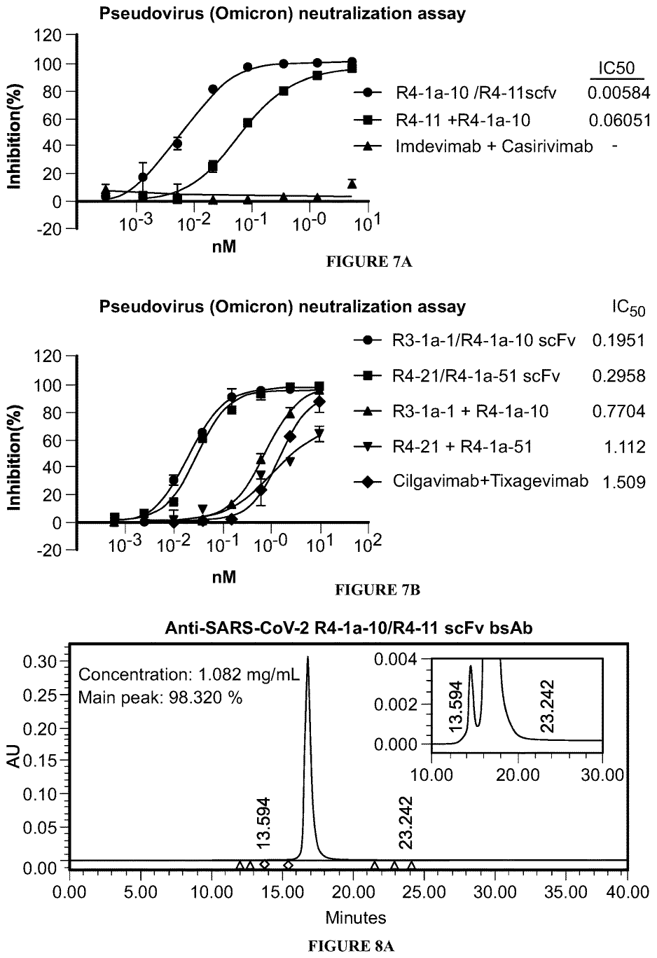

Neutralizing activity of bispecific antibody was evaluated by pseudo virus expressing SARS-CoV-2 Omicron spike protein and luciferase. Briefly, 293T-hACE2 cells (10e5 cells) were cultured in 24-well plate for 4 hours before the assay. The antibody samples were mixed with pseudo virus and incubated at room temperature for 30 minutes. The mixtures were added into 293T-hACE2 cells and cultured for 20 hours. Cells were lysed and viral entry into cells was quantified by Luciferase assay kit using a luminometer [model]. All assays were run in duplicate. Data was analyzed with GraphPad Prism and IC50 was calculated by nonlinear regression. Compared with the combination monoclonal antibodies, bispecific antibody R4-1a-10/R4-11 scFv showed a 10-fold increase in neutralizing activity (FIG. 7A). Compared with the combination monoclonal antibodies, bispecific antibody R3-1a-1/R4-1a-10 scFv showed a 4-fold increase in neutralizing activity (FIG. 7B).

### Example 9

### Evaluation of Protein Aggregation of Highly Concentrated Anti-SARS-CoV-2 Bispecific Antibody Leads by SEC-HPLC

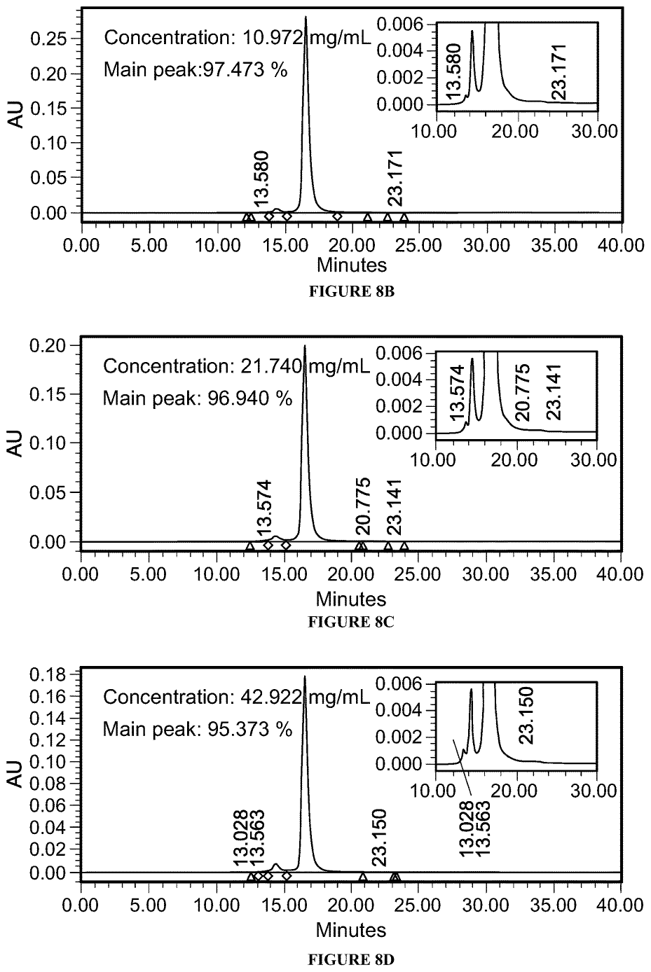

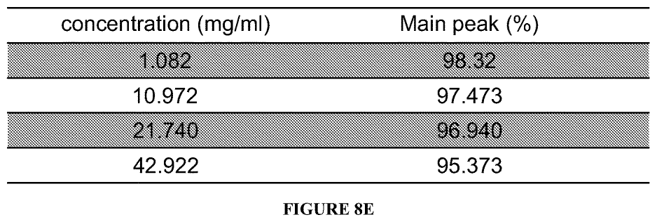

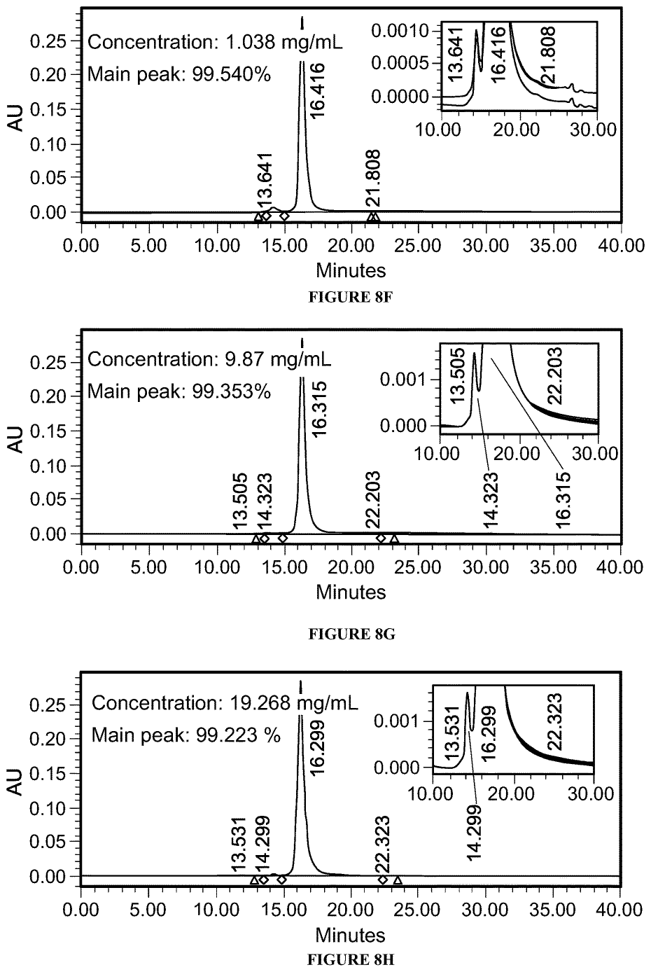

SEC-HPLC was applied to determine variants and aggregations of concentrated anti-SARS-CoV-2 bispecific antibody. SEC-HPLC was performed using a Waters ACQUITY Arc system with Waters 2489 UV/Vis detector. Samples were load into XBridge Protein BEH SEC Column (Waters, Cat #186007640) with isocratic 25 mM sodium phosphate, 200 mM NaCl, pH 6.8 as mobile phase buffer for SEC separation. The flow rate was 0.4 mL/min, and the sample injection amount was 40 μg. Peaks were detected by absorbance at 280 nm. Before injection onto the SEC column, all samples were filtrated with 0.22 μm filter (Millipore, Cat #SLGP003RB) to remove any precipitated protein material. Data were analyzed by Empower 3 software. In summary, the purity was greater than 99% of one-column Protein-A purified anti-SARS-CoV-2 R3-1a-1/R4-1a-10 scFv bispecific antibody at the concentration up to 40 mg/mL (FIG. 8A-8J).

### Example 10

### Evaluation of Protein Integrity of Anti-SARS-CoV-2 Bispecific Antibody by MCE-SDS-PAGE

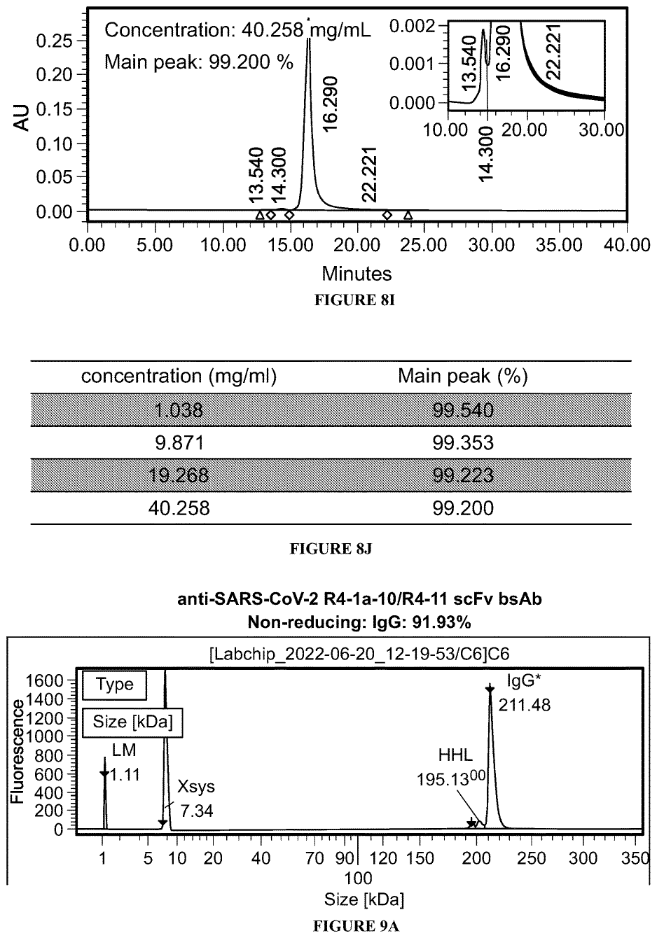

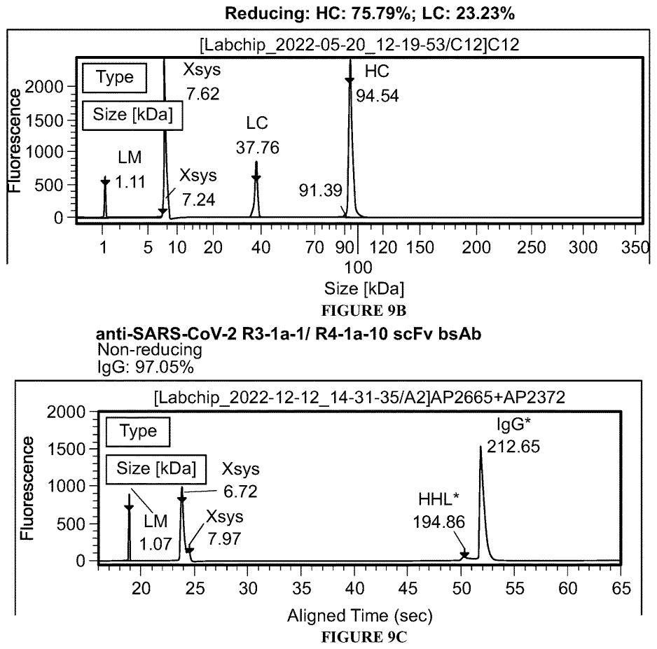

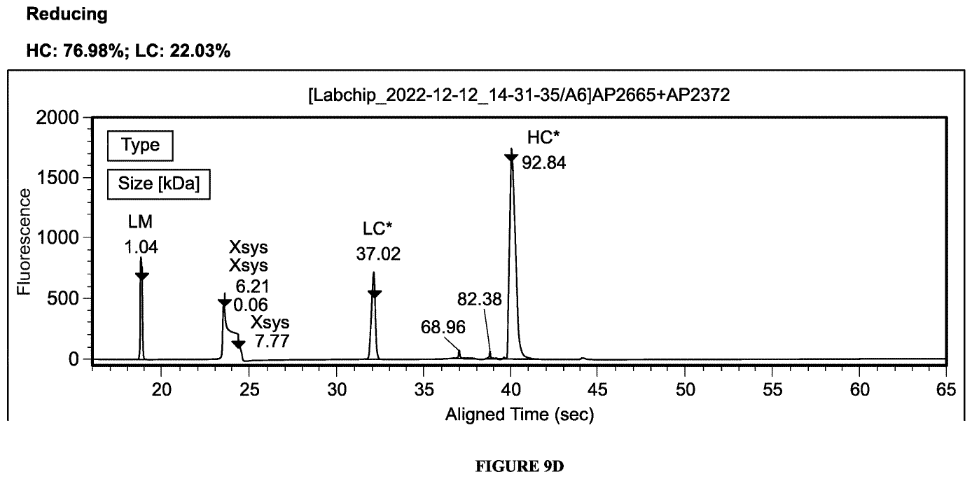

Anti-SARS-CoV-2 bispecific antibody was performed on a LabChip GXII instrument (Perkin Elmer, Inc.). The assay was carried out with “Protein Clear HR Assay” as manufacture's protocol. Briefly, 2.5 μl of protein sample at 1 mg/mL was mixed with 18 μl of sample buffer. The sample buffer was prepared by mixing 700 μl of Protein Clear HR sample buffer with either 24.5 μl of 1M DTT (for the reducing assay) or 24.5 μl of 0.25 M NEM (for the non-reducing assay). The samples were incubated at 70° C. for 10 min. After cooling to room temperature, 35 μl of water was added to each sample before loading onto the instrument. The samples were then analyzed using the Protein Clear HR Assay script. Meanwhile, the chip was prepared as instructed by the manufacturer and was maintained at 30° C. throughout the analysis. The excitation and detection wavelengths are 630 nm and 700 nm, respectively. The purity and integrity of one-column Protein-A purified anti-SARS-CoV-2 bsAb reach about 91% under non-reducing condition (FIGS. 9A-9B). In summary, the purity and integrity of one-column Protein-A purified anti-SARS-CoV-2 R3-1a-1/R4-1a-10 scFv bispecific antibody reach about 97% under non-reducing condition (FIGS. 9C and 9D).

Although the invention has been described with reference to the above examples, it will be understood that modifications and variations are encompassed within the spirit and scope of the invention. Accordingly, the invention is limited only by the following claims.

SEQUENCES

SEQ ID NO: 1: Anti-SARS-CoV-2 clone R4-11 heavy chain
QVQLVQSGAEVKKPGSSVKVSCKASGGAFSSYAISWVRQAPGQGLEWMGRIIPILGI
ANYAQKFQGRVTITADKSTSTAYMELSSLRSEDTAVYYCAGGSGYYYWGQGTTVT
VSS
SEQ ID NO: 2: Anti-SARS-CoV-2 clone R4-11 light chain
QSVLTQPPSVSEAPGQRVTISCSGSSSNIANNAVNWYQQLPGQAPKLLIFYDDLLPSW
VSDRFSGSKSGTSASLAISGLQSEDEADYYCAAWDDSLNGPVFGGGTKLTVLRQPVA
APSVTLFPPSSEELQANKATLVCLISDFYPGAVTVAWKADSSPVKAGVETTTPSKQSN
NKYAASSYLSLTPEQWKSHKSYSCQVTHEGSTVEKTVAPAECS
SEQ ID NO: 3: CDR-H1 of Anti-SARS-COV-2 clone R4-11
GGAFSSY
SEQ ID NO: 4: H2 of Anti-SARS-CoV-2 clone R4-11
IPILGI
SEQ ID NO: 5: CDR-H3 of Anti-SARS-CoV-2 clone R4-11
GSGYYY
SEQ ID NO: 6: CDR-L1 of Anti-SARS-CoV-2 clone R4-11
SGSSSNIANNAVN
SEQ ID NO: 7: CDR-L2 of Anti-SARS-CoV-2 clone R4-11
YDDLLPS
SEQ ID NO: 8: CDR-L3 of Anti-SARS-CoV-2 clone R4-11
AAWDDSLNGPV
SEQ ID NO: 9: Anti-SARS-COV-2 clone R4-1a-10 heavy chain
QVQLQQWGAGLLKPSETLSLTCAVYGGSFSGNYWSWIRQPPGKGLEWIGEINHSGS
TNYNPSLKSRVTISVDTSKNQFSLKLSSVTAADTAVYYCARGLLDYWGQGTLVTVSS
SEQ ID NO: 10: Anti-SARS-CoV-2 clone R4-1a-10 light chain
QSVLTQPSSVSAAPWQRVTISCSGSDSNIGNNPVNWYQHVPGKAPTLLIYYDNFLAS
KISDRFSGSKSGTSASLAISGLQSDDEADYYCASWDDSLNGVVFGGGTKLTVLRQPK
AAPSVTLFPPSSEELQANKATLVCLISDFYPGAVTVAWKADSSPVKAGVETTTPSKQS
NNKYAASSYLSLTPEQWKSHRSYSCQVTHEGSTVEKTVAPTECS
SEQ ID NO: 11: CDR-H1 of Anti-SARS-CoV-2 clone R4-1a-10
GGSFSGN
SEQ ID NO: 12: CDR-H2 of Anti-SARS-CoV-2 clone R4-1a-10
NHSGS
SEQ ID NO: 13: CDR-H3 of Anti-SARS-CoV-2 clone R4-1a-10
GLLDY
SEQ ID NO: 14: CDR-L1 of Anti-SARS-CoV-2 clone R4-1a-10
SGSDSNIGNNPVN
SEQ ID NO: 15: CDR-L2 of Anti-SARS-CoV-2 clone R4-1a-10
YDNFLAS
SEQ ID NO: 16: CDR-L3 of Anti-SARS-CoV-2 clone R4-1a-10
ASWDDSLNGVV
SEQ ID NO: 17: Anti-SARS-CoV-2 clone R4-1a-51 heavy chain
QVQLVQSGAEVKKPGASVKVSCKASGYTFTGYYMHWVRQAPGQGLEWMGRINPN
SGGTNYAQKFQGRVAMTRDTSISTAYMELSRLRSDDTAVYYCARGYMDYWGQGTP
VTVSS
SEQ ID NO: 18: Anti-SARS-CoV-2 clone R4-1a-51 light chain
QSVLTQPPSASGTPGQRVTISCSGSSSNIGSSTVNWYQQLPETAPKLLIYSNHQRPSGV
PDRFSGSKSGTSASLALSGLQSEDEAEYYCAAWDDSLNGVVFGGGTKLTVLGQPKA
APSVTLFPPSSEELQANKATLVCLISDFYPGAVTVAWKADSSPVKAGVETTTPSKQSN
NKYAASSYLSLTPEQWKSHKSYSCQVTHEGSTVEKTVAPTECS
SEQ ID NO: 19: CDR-H1 of Anti-SARS-CoV-2 clone R4-1a-51
GYTFTGY
SEQ ID NO: 20: CDR-H2 of Anti-SARS-CoV-2 clone R4-1a-51
NPNSGG
SEQ ID NO: 21: CDR-H3 of Anti-SARS-CoV-2 clone R4-1a-51
GYMDY
SEQ ID NO: 22: CDR-L1 of Anti-SARS-CoV-2 clone R4-1a-51
SGSSSNIGSSTVN
SEQ ID NO: 23: CDR-L2 of Anti-SARS-CoV-2 clone R4-1a-51
SNHQRPS
SEQ ID NO: 24: CDR-L3 of Anti-SARS-CoV-2 clone R4-1a-51
AAWDDSLNGVV
SEQ ID NO: 25: constant domain in heavy chain (IgG1)
ASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSCDKTHTCPPCPAP
ELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGK
SEQ ID NO: 26: SP1
METDTLLLWVLLLWVPGSTG
SEQ ID NO: 27: linker14
GSTSGSGKPGSGEGSTKG
SEQ ID NO: 28: (G4S)4 linker
GGGGSGGGGSGGGGSGGGGS
SEQ ID NO: 29: Anti-SARS-CoV-2 #R4-1a-10-#R4-11 scFv bsAb Heavy Chain
QVQLQQWGAGLLKPSETLSLTCAVYGGSFSGNYWSWIRQPPGKGLEWIGEINHSGS
TNYNPSLKSRVTISVDTSKNQFSLKLSSVTAADTAVYYCARGLLDYWGQGTLVTVSS
ASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSCDKTHTCPPCPAP
ELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGGSTSGSGKPGS
GEGSTKGQSVLTQPPSVSEAPGQRVTISCSGSSSNIANNAVNWYQQLPGQAPKLLIFY
DDLLPSWVSDRFSGSKSGTSASLAISGLQSEDEADYYCAAWDDSLNGPVFGGGTKLT
VLGGGGGSGGGGSGGGGSGGGGSQVQLVQSGAEVKKPGSSVKVSCKASGGAFSSY
AISWVRQAPGQGLEWMGRIIPILGIANYAQKFQGRVTITADKSTSTAYMELSSLRSED
TAVYYCAGGSGYYYWGQGTTVTVSSA
SEQ ID NO: 30: Anti-SARS-CoV-2 R4-11-scFv
QSVLTQPPSVSEAPGQRVTISCSGSSSNIANNAVNWYQQLPGQAPKLLIFYDDLLPSW
VSDRFSGSKSGTSASLAISGLQSEDEADYYCAAWDDSLNGPVFGGGTKLTVLGGGG
GSGGGGSGGGGSGGGGSQVQLVQSGAEVKKPGSSVKVSCKASGGAFSSYAISWVR
QAPGQGLEWMGRIIPILGIANYAQKFQGRVTITADKSTSTAYMELSSLRSEDTAVYY
CAGGSGYYYWGQGTTVTVSSA
SEQ ID NO: 31: Anti-SARS-CoV-2 #R4-11#R4-1a-10 scFv bsAb Heavy Chain
QVQLVQSGAEVKKPGSSVKVSCKASGGAFSSYAISWVRQAPGQGLEWMGRIIPILGI
ANYAQKFQGRVTITADKSTSTAYMELSSLRSEDTAVYYCAGGSGYYYWGQGTTVT
VSSASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTCPPCP
APELLGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQP
REPQVYTLPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGGGGGSGGGGS
GGGGSGGGGSQSVLTQPSSVSAAPWQRVTISCSGSDSNIGNNPVNWYQHVPGKAPT
LLIYYDNFLASKISDRFSGSKSGTSASLAISGLQSDDEADYYCASWDDSLNGVVFGG
GTKLTVLGGGGSGGGGSGGGGSGGGGSQVQLQQWGAGLLKPSETLSLTCAVYGGS
FSGNYWSWIRQPPGKGLEWIGEINHSGSTNYNPSLKSRVTISVDTSKNQFSLKLSSVT
AADTAVYYCARGLLDYWGQGTLVTVSSA
SEQ ID NO: 32: Anti-SARS-CoV-2 R4-1a-10-scFv
QSVLTQPSSVSAAPWQRVTISCSGSDSNIGNNPVNWYQHVPGKAPTLLIYYDNFLAS
KISDRESGSKSGTSASLAISGLQSDDEADYYCASWDDSLNGVVFGGGTKLTVLGGGG
SGGGGSGGGGSGGGGSQVQLQQWGAGLLKPSETLSLTCAVYGGSFSGNYWSWIRQ
PPGKGLEWIGEINHSGSTNYNPSLKSRVTISVDTSKNQFSLKLSSVTAADTAVYYCAR
GLLDYWGQGTLVTVSSA
SEQ ID NO: 33: Anti-SARS-CoV-2 clone R3-1a-1 heavy chain
QVQLVQSGAEVKKPGSSVKVSCKASGGTFSSYAISWVRQAPGQGLEWMGGIIPILGI
ANYAQKFQGRVTITADKSTSTAYMELSSLRSEDTAVYYCARDGGGSSGYHDYWGQ
GTLVTVSS
SEQ ID NO: 34: Anti-SARS-CoV-2 clone R3-1a-1 light chain
NFMLTQPHSVSESPGKTVTISCTRSSGSIASKYVQWYQQRPGNSPTTVIYEDDQRPSG
VPDRFSASIDSSANTASLTISGLKPEDEADYYCQSYDDNTAVFGGGTHLTVLGQPKA
APSVTLFPPSSEELQANKATLVCLVSDFYPGAVTVAWKADGSPVKVGVETTKPSKQS
NNKYAASSYLSLTPEQWKSHRSYSCRVTHEGSTVEKTVAPAECS
SEQ ID NO: 35: CDR-H1 of Anti-SARS-CoV-2 clone R3-1a-1
GGTFSSY
SEQ ID NO: 36: CDR-H2 of Anti-SARS-CoV-2 clone R3-1a-1
IPILGI
SEQ ID NO: 37: CDR-H3 of Anti-SARS-CoV-2 clone R3-1a-1
DGGGSSGYHDY
SEQ ID NO: 38: CDR-L1 of Anti-SARS-CoV-2 clone R3-1a-1
TRSSGSIASKYVQ
SEQ ID NO: 39: CDR-L2 of Anti-SARS-CoV-2 clone R3-1a-1
EDDQRPS
SEQ ID NO: 40: CDR-L3 of Anti-SARS-CoV-2 clone R3-1a-1
QSYDDNTAV
SEQ ID NO: 41: Anti-SARS-CoV-2 clone R4-21 heavy chain
QVQLVQSGAEVKKPGESLKISCKGSGYSFTSYWIGWVRQMPGKGLEWMGIIYPGDS
DTRYSPSFQGQVTISADKSISTAYLQWSSLKASDTAMYYCATQTPYYDSSDYYLGDA
FDIWGQGTMVTVSS
SEQ ID NO: 42: Anti-SARS-CoV-2 clone R4-21 light chain
QSALTQPASVSGSPGQSITISCTGTSSDVGGYNYVSWYQQHPGKAPKLIIYDVRNRPS
GVSNRFSGSKSGNTASLTISGLQAEDEADYYCGSYTSSSTLGFGGGTKLTVLGQPKA
APSVTLFPPSSEELQANKATLVCLISDFYPGAVTVAWKADSSPVKAGVETTTPSKQSN
NKYAASSYLSLTPEQWKSHKSYSCQVTHEGSTVEKTVAPTECS
SEQ ID NO: 43: CDR-H1 of Anti-SARS-CoV-2 clone R4-21
GYSFTSY
SEQ ID NO: 44: CDR-H2 of Anti-SARS-CoV-2 clone R4-21
YPGDSD
SEQ ID NO: 45: CDR-H3 of Anti-SARS-CoV-2 clone R4-21
QTPYYDSSDYYLGDAFDI
SEQ ID NO: 46: CDR-L1 of Anti-SARS-CoV-2 clone R4-21
TGTSSDVGGYNYVS
SEQ ID NO: 47: CDR-L2 of Anti-SARS-CoV-2 clone R4-21
DVRNRPS
SEQ ID NO: 48: CDR-L3 of Anti-SARS-CoV-2 clone R4-21
GSYTSSSTLG
SEQ ID NO: 49: Anti-SARS-CoV-2 #R3-1a-1-# R4-1a-10 scFv bsAb Heavy Chain
QVQLVQSGAEVKKPGSSVKVSCKASGGTFSSYAISWVRQAPGQGLEWMGGIIPILGI
ANYAQKFQGRVTITADKSTSTAYMELSSLRSEDTAVYYCARDGGGSSGYHDYWGQ
GTLVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKVEPKSCDKT
HTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNGQPENNYKT
TPPVLDSDGSFFLYSKLTVDKSRWQQGNVFSCSVMHEALHNHYTQKSLSLSPGGGG
GSGGGGSGGGGSGGGGSQSVLTQPSSVSAAPWQRVTISCSGSDSNIGNNPVNWYQH
VPGKAPTLLIYYDNFLASKISDRESGSKSGTSASLAISGLQSDDEADYYCASWDDSLN
GVVFGGGTKLTVLGGGGSGGGGSGGGGSGGGGSQVQLQQWGAGLLKPSETLSLTC
AVYGGSFSGNYWSWIRQPPGKGLEWIGEINHSGSTNYNPSLKSRVTISVDTSKNQFSL
KLSSVTAADTAVYYCARGLLDYWGQGTLVTVSSA
SEQ ID NO: 50: Anti-SARS-CoV-2 #R3-1a-1-# R4-1a-51 scFv bsAb Heavy Chain
QVQLVQSGAEVKKPGSSVKVSCKASGGTFSSYAISWVRQAPGQGLEWMGGIIPILGI
ANYAQKFQGRVTITADKSTSTAYMELSSLRSEDTAVYYCARDGGGSSGYHDYWGQ
GTLVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKVEPKSCDKT
HTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNGQPENNYKT
TPPVLDSDGSFFLYSKLTVDKSRWQQGNVFSCSVMHEALHNHYTQKSLSLSPGGGG
GSGGGGSGGGGSGGGGSQSVLTQPPSASGTPGQRVTISCSGSSSNIGSSTVNWYQQLP
ETAPKLLIYSNHQRPSGVPDRESGSKSGTSASLALSGLQSEDEAEYYCAAWDDSLNG
VVFGGGTKLTVLGGGGSGGGGSGGGGSGGGGSQVQLVQSGAEVKKPGASVKVSCK
ASGYTFTGYYMHWVRQAPGQGLEWMGRINPNSGGTNYAQKFQGRVAMTRDTSIST
AYMELSRLRSDDTAVYYCARGYMDYWGQGTPVTVSSA
SEQ ID NO: 51: Anti-SARS-CoV-2 #R4-21-# R4-1a-10 scFv bsAb Heavy Chain
QVQLVQSGAEVKKPGESLKISCKGSGYSFTSYWIGWVRQMPGKGLEWMGIIYPGDS
DTRYSPSFQGQVTISADKSISTAYLQWSSLKASDTAMYYCATQTPYYDSSDYYLGDA
FDIWGQGTMVTVSSASTKGPSVFPLAPSSKSTSGGTAALGCLVKDYFPEPVTVSWNS
GALTSGVHTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALP
APIEKTISKAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYTQKSLSL
SPGGGGGSGGGGSGGGGSGGGGSQSVLTQPSSVSAAPWQRVTISCSGSDSNIGNNPV
NWYQHVPGKAPTLLIYYDNFLASKISDRFSGSKSGTSASLAISGLQSDDEADYYCAS
WDDSLNGVVFGGGTKLTVLGGGGSGGGGSGGGGSGGGGSQVQLQQWGAGLLKPS
ETLSLTCAVYGGSFSGNYWSWIRQPPGKGLEWIGEINHSGSTNYNPSLKSRVTISVDT
SKNQFSLKLSSVTAADTAVYYCARGLLDYWGQGTLVTVSSA
SEQ ID NO: 52: Anti-SARS-CoV-2 #R4-21-# R4-1a-51 scFv bsAb Heavy Chain
QVQLVQSGAEVKKPGESLKISCKGSGYSFTSYWIGWVRQMPGKGLEWMGIIYPGDS
DTRYSPSFQGQVTISADKSISTAYLQWSSLKASDTAMYYCATQTPYYDSSDYYLGDA
FDIWGQGTMVTVSSASTKGPSVFPLAPSSKSTSGGTAALGCLVKDYFPEPVTVSWNS
GALTSGVHTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALP
APIEKTISKAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYTQKSLSL
SPGGGGGSGGGGSGGGGSGGGGSQSVLTQPPSASGTPGQRVTISCSGSSSNIGSSTVN
WYQQLPETAPKLLIYSNHQRPSGVPDRFSGSKSGTSASLALSGLQSEDEAEYYCAAW
DDSLNGVVFGGGTKLTVLGGGGSGGGGSGGGGSGGGGSQVQLVQSGAEVKKPGAS
VKVSCKASGYTFTGYYMHWVRQAPGQGLEWMGRINPNSGGTNYAQKFQGRVAMT
RDTSISTAYMELSRLRSDDTAVYYCARGYMDYWGQGTPVTVSSA
SEQ ID NO: 53: Anti-SARS-CoV-2 R4-1a-51-scFv
QSVLTQPPSASGTPGQRVTISCSGSSSNIGSSTVNWYQQLPETAPKLLIYSNHQRPSGV
PDRFSGSKSGTSASLALSGLQSEDEAEYYCAAWDDSLNGVVFGGGTKLTVLGGGGS
GGGGSGGGGSGGGGSQVQLVQSGAEVKKPGASVKVSCKASGYTFTGYYMHWVRQ
APGQGLEWMGRINPNSGGTNYAQKFQGRVAMTRDTSISTAYMELSRLRSDDTAVY
YCARGYMDYWGQGTPVTVSSA

### REFERENCES

Renn, A., Fu, Y., Hu, X., Hall, M. D. & Simeonov, A. Fruitful neutralizing antibody pipeline brings hope to defeat SARS-Cov-2. Trends Pharmacol. Sci. 41, 815-829 (2020).
Shanmugaraj, B., Siriwattananon, K., Wangkanont, K. & Phoolcharoen, W. Perspectives on monoclonal antibody therapy as potential therapeutic intervention for coronavirus disease-19 (COVID-19). Asian Pac. J. Allergy Immunol. 38, 10-18 (2020).
Ecker, D. M. & Seymour, P. in CPhI Annual Report 2020: Postulating the Post-COVID Pharma Paradigm, 43-49 (Informamarkets, 2020).

1. An antibody or antigen binding fragment thereof comprising:
(i) a VH region comprising complementarity-determining region (CDR)-H1, CDR-H2, and CDR-H3, wherein CDR-H1 is set forth in SEQ ID NO:3, CDR-H2 is set forth in SEQ ID NO:4, and CDR-H3 is set forth in SEQ ID NO:5 or sequences having 90% identity to SEQ ID NO:3, 4 or 5 and the antigen binding specificity thereof; and a VL region comprising CDR-L1, CDR-L2, and CDR-L3, wherein CDR-L1 is set forth in SEQ ID NO:6, CDR-L2 is set forth in SEQ ID NO:7, and CDR-L3 is set forth in SEQ ID NO:8 or sequences having 90% identity to SEQ ID NO:6, 7 or 8 and the antigen binding specificity thereof;
(ii) a VH region comprising CDR-H1, CDR-H2, and CDR-H3, wherein CDR-H1 is set forth in SEQ ID NO:11, CDR-H2 is set forth in SEQ ID NO:12, and CDR-H3 is set forth in SEQ ID NO:13 or sequences having 90% identity to SEQ ID NO:11, 12 or 13 and the antigen binding specificity thereof; and a VL region comprising CDR-L1, CDR-L2, and CDR-L3, wherein CDR-L1 is set forth in SEQ ID NO:14, CDR-L2 is set forth in SEQ ID NO:15, and CDR-L3 is set forth in SEQ ID NO:16 or sequences having 90% identity to SEQ ID NO:14, 15 or 16 and the antigen binding specificity thereof;
(iii) a VH region comprising CDR-H1, CDR-H2, and CDR-H3, wherein CDR-H1 is set forth in SEQ ID NO:19, CDR-H2 is set forth in SEQ ID NO:20, and CDR-H3 is set forth in SEQ ID NO:21 or sequences having 90% identity to SEQ ID NO:19, 20 or 21 and the antigen binding specificity thereof; and a VL region comprising CDR-L1, CDR-L2, and CDR-L3, wherein CDR-L1 is set forth in SEQ ID NO:22, CDR-L2 is set forth in SEQ ID NO:23, and CDR-L3 is set forth in SEQ ID NO:24 or sequences having 90% identity to SEQ ID NO:22, 23 or 24 and the antigen binding specificity thereof,
(iv) a VH region comprising CDR-H1, CDR-H2, and CDR-H3, wherein CDR-H1 is set forth in SEQ ID NO:35, CDR-H2 is set forth in SEQ ID NO:36, and CDR-H3 is set forth in SEQ ID NO:37 or sequences having 90% identity to SEQ ID NO:35, 36 or 37 and the antigen binding specificity thereof; and a VL region comprising CDR-L1, CDR-L2, and CDR-L3, wherein CDR-L1 is set forth in SEQ ID NO:38, CDR-L2 is set forth in SEQ ID NO:39, and CDR-L3 is set forth in SEQ ID NO:40 or sequences having 90% identity to SEQ ID NO:38, 39 or 40 and the antigen binding specificity thereof; or
(v) a VH region comprising CDR-H1, CDR-H2, and CDR-H3, wherein CDR-H1 is set forth in SEQ ID NO:43, CDR-H2 is set forth in SEQ ID NO:44, and CDR-H3 is set forth in SEQ ID NO:45 or sequences having 90% identity to SEQ ID NO:43, 44 or 45 and the antigen binding specificity thereof; and a VL region comprising CDR-L1, CDR-L2, and CDR-L3, wherein CDR-L1 is set forth in SEQ ID NO:46, CDR-L2 is set forth in SEQ ID NO:47, and CDR-L3 is set forth in SEQ ID NO:48 or sequences having 90% identity to SEQ ID NO:46, 47 or 48 and the antigen binding specificity thereof; wherein the antibody or antigen binding fragment binds to SARS-CoV-2 spike protein.

2. The antibody or antigen binding fragment of claim 1, wherein:
(i) the VL region has an amino acid sequence having at least 80% identity to SEQ ID NO:1 and an antigen binding specificity of SEQ ID NOs:3-5 and the VL region has an amino acid sequence having at least 80% identity to SEQ ID NO:2 and an antigen binding specificity of SEQ ID NOs:6-8;
(ii) the VH region has an amino acid sequence having at least 80% identity to SEQ ID NO:9 and an antigen binding specificity of SEQ ID NOs:11-13 and the VL region has an amino acid sequence having at least 80% identity to SEQ ID NO:10 and an antigen binding specificity of SEQ ID NOs:14-16;
(iii) the VH region has an amino acid sequence having at least 80% identity to SEQ ID NO:17 and an antigen binding specificity of SEQ ID NOs:19-21 and the VL region has an amino acid sequence having at least 80% identity to SEQ ID NO:18 and an antigen binding specificity of SEQ ID NOs:22-24;
(iv) the VH region has an amino acid sequence having at least 80% identity to SEQ ID NO:33 and an antigen binding specificity of SEQ ID NOs:35-37 and the VL region has an amino acid sequence having at least 80% identity to SEQ ID NO:34 and an antigen binding specificity of SEQ ID NOs:38-40; or
(v) the VH region has an amino acid sequence having at least 80% identity to SEQ ID NO:41 and an antigen binding specificity of SEQ ID NOs:43-45 and the VL region has an amino acid sequence having at least 80% identity to SEQ ID NO:42 and an antigen binding specificity of SEQ ID NOs:46-48.

3. The antibody or antigen binding fragment of claim 1, wherein:
(i) the VH region is set forth in SEQ ID NO:1 and the VL region is set forth in SEQ ID NO:2 or a sequence having 90% identity thereto and the binding specificity thereof;
(ii) the VH region is set forth in SEQ ID NO:9 and the VL region is set forth in SEQ ID NO:10 or a sequence having 90% identity thereto and the binding specificity thereof;
(iii) the VH region is set forth in SEQ ID NO:17 and the VL region is set forth in SEQ ID NO:18 or a sequence having 90% identity thereto and the binding specificity thereof;
(iv) the VH region is set forth in SEQ ID NO:33 and the VL region is set forth in SEQ ID NO:34 or a sequence having 90% identity thereto and the binding specificity thereof; or
(v) the VH region is set forth in SEQ ID NO:41 and the VL region is set forth in SEQ ID NO:42 or a sequence having 90% identity thereto and the binding specificity thereof.

4. The antibody or antigen binding fragment of claim 1, wherein the antibody comprises an Fc domain.

5. The antibody or antigen binding fragment of claim 4, wherein the Fc domain is an IgG domain, an IgE domain, an IgM domain, and IgD domain, an IgA domain, or an IgY domain.

6. The antibody or antigen binding fragment of claim 5, wherein the IgG domain is an IgG1 domain, an IgG2 domain, an IgG3 domain, or an IgG4 domain.

7. The antibody or antigen binding fragment of claim 6, wherein the IgG1 domain comprises an amino acid sequence of SEQ ID NO:25.

8. A bispecific antibody comprising a first antigen binding region that binds to a first SARS-CoV-2 spike protein antigen and a second antigen binding region that binds to a second SARS-CoV-2 spike protein antigen.

9. The bispecific antibody of claim 8, having the amino acid sequence as set forth in SEQ ID NO:29, 31, 49, 50, 51 or 52, or a sequence having 90% identity thereto and the binding specificity thereof.

10. The bispecific antibody of claim 8, wherein the antibody comprises an Fc domain.

11. The bispecific antibody of claim 10, wherein the Fc domain is an IgG domain, an IgE domain, an IgM domain, and IgD domain, an IgA domain, or an IgY domain.

12. The bispecific antibody of claim 10, wherein the Fc domain is an IgG domain.

13. The bispecific antibody of claim 12, wherein the IgG domain is an IgG1 domain, an IgG2 domain, an IgG3 domain, or an IgG4 domain.

14. The bispecific antibody of claim 10, wherein a scFv is linked to the C-terminus of the Fc domain.

15. The bispecific antibody of claim 14, wherein the antibody comprises a linker between the Fab domain and the scFv domain.

16. The bispecific antibody of claim 15, wherein the Fab fragment is linked to the N-terminus of the Fc domain.

17. A pharmaceutical composition comprising the antibody or an antigen binding fragment thereof of claim 1.

18. The pharmaceutical composition of claim 17, further comprising a pharmaceutically acceptable carrier conjugated to the C-terminus of one or more polypeptides of the antibody or antigen binding fragment thereof.

19. An isolated amino acid sequence as set forth in SEQ ID NOs:1-53.

20. A pharmaceutical composition comprising the bispecific antibody of claim 8.
